(19) United States
(12) Patent Application Publication
(10) Pub. No.: US 2012/0071448 A1
(43) Pub. Date: Mar. 22, 2012

(54) S1P3 Receptor Inhibitors for Treating Conditions of the Eye

(75) Inventors: John E. Donello, Dana Point, CA (US); Richard L. Beard, Newport Beach, CA (US); Mohammed I. Dibas, Laguna Niguel, CA (US)

(73) Assignee: Allergan, Inc., Irvine, CA (US)

(21) Appl. No.: 13/318,583

(22) PCT Filed: May 4, 2010
(86) PCT No.: PCT/US2010/033553

§ 371 Date: Dec. 2, 2011

Related U.S. Application Data
(60) Provisional application No. 61/175,763, filed on May 5, 2009.

(51) Int. Cl. A61K 31/675; A61P 27/02; A61K 31/4418

(52) U.S. Cl. 514/89; 514/357

(57) ABSTRACT

Disclosed herein are compositions and methods for treating conditions of the eye using S1P3 receptor inhibitors.

### CROSS-REFERENCE

This application claims the benefit of U.S. Provisional Patent Application Ser. No. 61/175,763, filed on May 5, 2009, the entire disclosure of which is incorporated herein by this specific reference.

### INTRODUCTION

Disclosed herein is a method for treating conditions of the eye, the method comprising administering to a patient in need of such treatment an S1P3 receptor inhibitor.

### SUMMARY OF THE INVENTION

The present invention provides a method for treating a condition of the eye, the method comprising the step of administering to a patient in need of such treatment an S1P3 receptor inhibitor.

In one embodiment, the S1P3 receptor inhibitor comprises an anti-S1P3 receptor polyclonal, monoclonal, humanized, bispecific, or heteroconjugate antibody.

In another embodiment, the present invention provides a method for treating conditions of the eye, the method comprising the step of administering to a patient in need of such treatment a compound represented by the general formula I

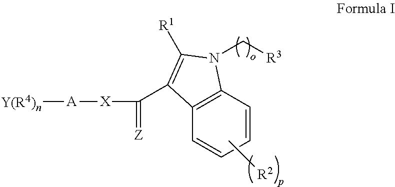

or a pharmaceutically acceptable salt thereof, wherein

X is NR⁵, O, S;

Z is O or S;

n is 0 or an integer of from 1 to 4;

o is 0 or an integer of from 1 to 3;

p is 0 or an integer of from 1 to 4;

A is (C(R⁵)₂)ₘ, wherein
m is 0 or an integer of from 1 to 6;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl groups;

Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

R¹, R², R³, R⁴ are selected from the group consisting of hydrogen; straight or branched chain alkyl having 1 to 12 carbons; cycloalkyl having 3 to 6 carbons; alkenyl having 2 to 6 carbons and 1 or 2 double bonds; alkynyl having 2 to 6 carbons and 1 or 2 triple bonds; aryl wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur; halo; C₁ to C₁₂ haloalkyl; hydroxyl; C₁ to C₁₂ alkoxy; C₃ to C₂₀ arylalkyloxy; C₁ to C₁₂ alkylcarbonyl; formyl; oxycarbonyl; carboxy; C₁ to C₁₂ alkyl carboxylate; C₁ to C₁₂ alkyl amide; aminocarbonyl; amino; cyano; diazo; nitro; thio; sulfoxyl; sulfonyl groups; or a group selected from the group consisting of

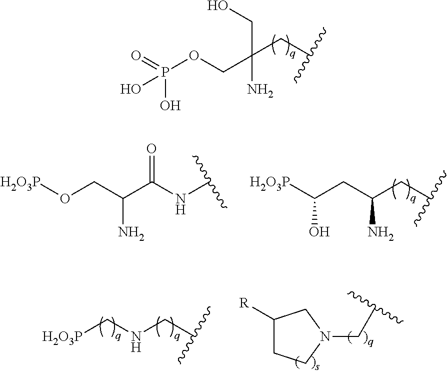

wherein R is CO₂H or PO₃H₂, p is an integer of 1 or 2 and q is 0 or an integer of 1 to 5 and s is 0 or an integer of 1 or 2; provided that, if Y is phenyl, it must be substituted with at least one R⁴ group that is not hydrogen.

In another embodiment, in Formula I, Z is O.

In another embodiment, in Formula I, Y is a phenyl group, or a heterocyclic aryl group selected from the group consisting of pyridyl, thienyl, furyl, pyradizinyl, pyrimidinyl, pyrazinyl, thiazolyl, oxazolyl, and imidazolyl.

In another embodiment, in Formula I, said Y is independently selected from the group consisting of phenyl, pyridine, pyrazine, pyridazine, pyrimidine, triazine, thiophene, furan, thiazole, thiadiazole, isothiazole, oxazole, oxadiazole, isooxazole, naphthalene, quinoline, tetralin, chroman, thiochroman, tetrahydroquinoline, dihydronaphthalene, tetrahydronaphthalen, chromene, thiochromene, dihydroquinoline, indan, dihydrobenzofuran, dihydrobenzothiophene, indene, benzofuran, benzothiophene, coumarin and coumarinone, wherein said aryl is unsubstituted or is substituted with one or two alkyl, alkenyl, alkynyl, aryl, halo, haloalkyl, hydroxyl, alkoxyl, alkylcarbonyl, formyl, oxycarbonyl, carboxyl, alkyl carboxylate, alkyl amide, aminocarbonyl, amino, cyano, diazo, nitro, thio, sulfoxyl, or sulfonyl groups.

In another embodiment, in Formula I, Y is phenyl.

In another embodiment, in Formula I, A is CH₂.

In another embodiment, in Formula I, X is NH.

In another embodiment, in Formula I, n is 0 or an integer of 1 or 2 and R⁴ is selected from the group consisting of methyl, methoxy, fluoro and chloro.

In another embodiment, in Formula I, R¹ is selected from the group consisting of hydrogen, methyl, ethyl and i-propyl.

In another embodiment, in Formula I, R³ is selected from the group consisting of methyl, butyl, phenyl, benzyl, pyridyl, furanylmethylenyl, thienyl and thienyl methylenyl.

In another embodiment, in Formula I, p is 0 or p is 1 and R² is selected from the group consisting of hydroxyl, methoxy, nitro, amino, acetamido and benzyloxy.

In another embodiment, in Formula I, p is 1 and R² is a 5-hydroxy group; R¹ is selected from the group consisting of methyl, ethyl, i-propyl and phenyl; R³ is selected from the group consisting of benzyl, thienylmethylenyl and furanylmethylenyl; n is 1 or 2 and R⁴ is selected from the group consisting of methoxy and fluoro.

In another embodiment, the compound of Formula I is selected from the group consisting of
1-Benzyl-5-hydroxy-2-methyl-1H-indole-3-carboxylic Acid, 3,5-Difluorobenzylamide;
5-Hydroxy-2-methyl-1-thiophen-2-ylmethyl-1H-indole-3-carboxylic Acid, 3,4-Difluorobenzylamide;
1-Butyl-5-hydroxy-2-methyl-1H-indole-3-carboxylic Acid, 3,5-Difluoro-benzylamide;
1-Furan-2-ylmethyl-5-hydroxy-2-methyl-1H-indole-3-carboxylic Acid, 3,4-Difluorobenzylamide;
5-Hydroxy-2-methyl-1-thiophen-2-ylmethyl-1H-indole-3-carboxylic Acid, 3,5-Difluorobenzylamide;
1-Furan-2-ylmethyl-5-hydroxy-2-methyl-1H-indole-3-carboxylic Acid 3,5-Difluorobenzylamide;
1-Benzyl-5-hydroxy-2-methyl-1H-indole-3-carboxylic Acid. 3,4-Difluoro-benzylamide;
5-Hydroxy-2-methyl-1-thiophen-2-ylmethyl-1H-indole-3-carboxylic Acid, 3-Fluorobenzylamide;
5-Hydroxy-2-methyl-1-thiophen-2-ylmethyl-1H-indole-3-carboxylic Acid, Benzylamide;
5-Hydroxy-2-methyl-1-thiophen-2-ylmethyl-1H-indole-3-carboxylic Acid, 3-Methoxybenzylamide;
1-Butyl-5-hydroxy-2-methyl-1H-indole-3-carboxylic Acid, 3-Methoxy-benzylamide;
5-Hydroxy-2-methyl-1-thiophen-2-ylmethyl-1H-indole-3-carboxylic Acid, 4-Fluorobenzylamide;
5-Hydroxy-2-methyl-1-thiophen-2-ylmethyl-1H-indole-3-carboxylic Acid, 4-Methylbenzylamide;
5-Hydroxy-2-methyl-1-thiophen-2-ylmethyl-1H-indole-3-carboxylic Acid, 3-Chlorobenzylamide;
5-Hydroxy-2-methyl-1-thiophen-2-ylmethyl-1H-indole-3-carboxylic Acid, 4-Chlorobenzylamide;
5-Hydroxy-2-methyl-1-thiophen-2-ylmethyl-1H-indole-3-carboxylic Acid, 2-methoxybenzylamide;
1-Benzyl-2-ethyl-5-hydroxy-1H-indole-3-carboxylic Acid, 3,4-Difluoro-benzylamide;
1-Benzyl-2-ethyl-5-hydroxy-1H-indole-3-carboxylic Acid, 3-Methoxy-benzylamide;
1-Benzyl-5-hydroxy-2-isopropyl-1H-indole-3-carboxylic Acid, 3,4-Difluorobenzamide;
5-Hydroxy-2-methyl-1-phenyl-1H-indole-3-carboxylic Acid 3,4-Difluoro-benzylamide;
5-Hydroxy-2-methyl-1-pyridin-2-yl-1H-indole-3-carboxylic Acid 3,4-Difluoro-benzylamide;
5-Hydroxy-2-methyl-1-thiophen-2-yl-1H-indole-3-carboxylic Acid 3,4-Difluorobenzylamide;
1-Benzyl-2-ethyl-5-hydroxy-1H-indole-3-carboxylic Acid 3,5-Difluoro-benzylamide;
1-Benzyl-5-hydroxy-2-isopropyl-1H-indole-3-carboxylic Acid, 3,5-difluorobenzylamide;
1-Benzyl-5-hydroxy-2-isopropyl-1H-indole-3-carboxylic Acid, 3-methoxybenzylamide; and
1-Benzyl-5-hydroxy-2-phenyl-1H-indole-3-carboxylic Acid, 3,5-Difluoro-benzylamide.

In another embodiment, the compound of Formula I is selected from the group consisting of
1-Benzyl-5-hydroxy-2-methyl-1H-indole-3-carboxylic Acid, 3,5-Difluorobenzylamide;
1-Furan-2-ylmethyl-5-hydroxy-2-methyl-1H-indole-3-carboxylic Acid 3,5-Difluorobenzylamide;
5-Hydroxy-2-methyl-1-thiophen-2-ylmethyl-1H-indole-3-carboxylic Acid, 3-Methoxybenzylamide;
1-Benzyl-2-ethyl-5-hydroxy-1H-indole-3-carboxylic Acid, 3,4-Difluoro-benzylamide;
1-Benzyl-2-ethyl-5-hydroxy-1H-indole-3-carboxylic Acid 3,5-Difluoro-benzylamide;
1-Benzyl-5-hydroxy-2-isopropyl-1H-indole-3-carboxylic Acid, 3,5-difluorobenzylamide;
1-Benzyl-5-hydroxy-2-isopropyl-1H-indole-3-carboxylic Acid, 3-methoxybenzylamide; and
1-Benzyl-5-hydroxy-2-phenyl-1H-indole-3-carboxylic Acid, 3,5-Difluoro-benzylamide.

In another embodiment, the present invention provides a method for treating conditions of the eye, the method comprising the step of administering to a patient in need of such treatment a compound represented by the general formula II:

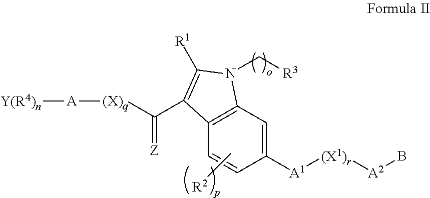

wherein:

R¹, R², R³ and R⁴ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, carbocyclic hydrocarbon groups having from 3 to 20 carbon atoms, heterocyclic groups having up to 20 carbon atoms and at least one of oxygen, nitrogen and/or sulfur in the ring, halo, C₁ to C₁₂ haloalkyl, hydroxyl, C₁ to C₁₂ alkoxy, C₃ to C₂₀ arylalkyloxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, and sulfonyl groups;

X and X¹ are independently selected from the group consisting of NR⁵, O and S;

R⁵ is hydrogen, an alkyl group of 1 to 10 carbons, a cycloalkyl group of 5 to 10 carbons, phenyl or lower alkylphenyl;

Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

Z is O or S;

n is 0 or an integer of from 1 to 5;

o is 0 or an integer of from 1 to 3;

p is 0 or an integer of from 1 to 3;

q is 0 or 1;

r is 0 or 1;

A, A¹ and A² are independently selected from the group consisting of (CH₂)ᵥ wherein v is 0 or an integer of from 1 to 12, branched chain alkyl having 3 to 12 carbons, cycloalkyl having 3 to 12 carbons, alkenyl having 2 to 10 carbons and 1-3 double bonds and alkynyl having 2 to 10 carbons and 1 to 3 triple bonds;

B is selected from the group consisting of hydrogen, OR⁶, COOR⁷, NR⁸R⁹, CONR⁸R⁹, COR¹⁰, CH═NOR¹¹, CH═NNR¹²R¹³, wherein R⁶, R⁷, R¹⁹ and R¹¹ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, R⁸, R⁹, R¹² and R¹³ are are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, or R⁸ and R⁹ and/or R¹² and R¹³, together, can form a divalent carbon radical of 2 to 5 carbons to form a heterocyclic ring with nitrogen, wherein any of R⁶, R⁷, R⁸, R⁹, R¹⁰, R¹¹, R¹² or R¹³ may be substituted with one or more halogen, hydroxy, alkyloxy, cyano, nitro, mercapto or thiol radical; provided however, when v is 0, and r is 0, B is not hydrogen; or B is a carbocyclic hydrocarbon group having from 3 to 20 carbon atoms, or a heterocyclic group having up to 20 carbon atoms and at least one of oxygen, nitrogen and/or sulfur in the ring, and wherein when said B is a carbocyclic or heterocyclic group B may be bonded to A² at any position, or a pharmaceutically acceptable salt of said compound.

In another embodiment, the compound of Formula II is selected from the group consisting of the following compounds:

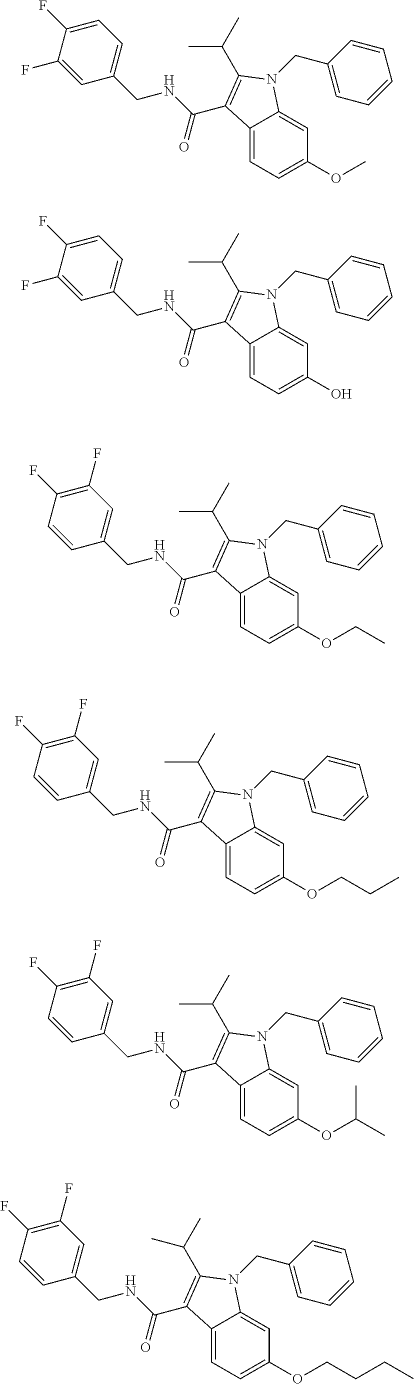

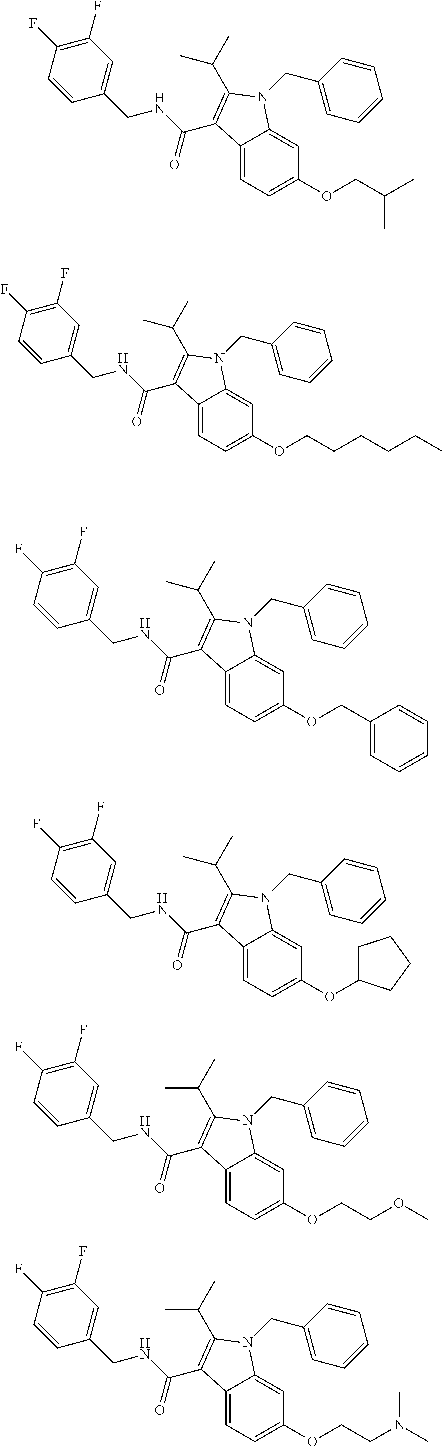

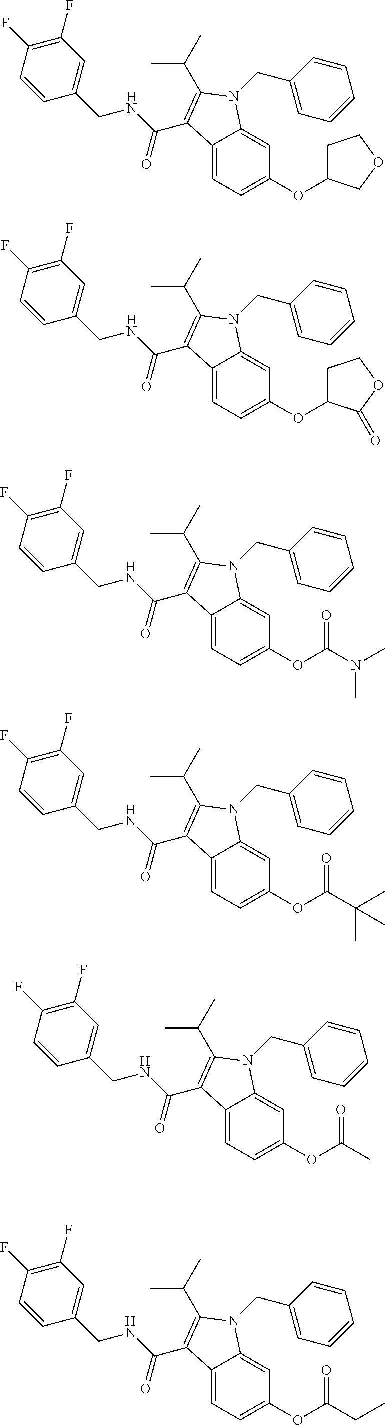

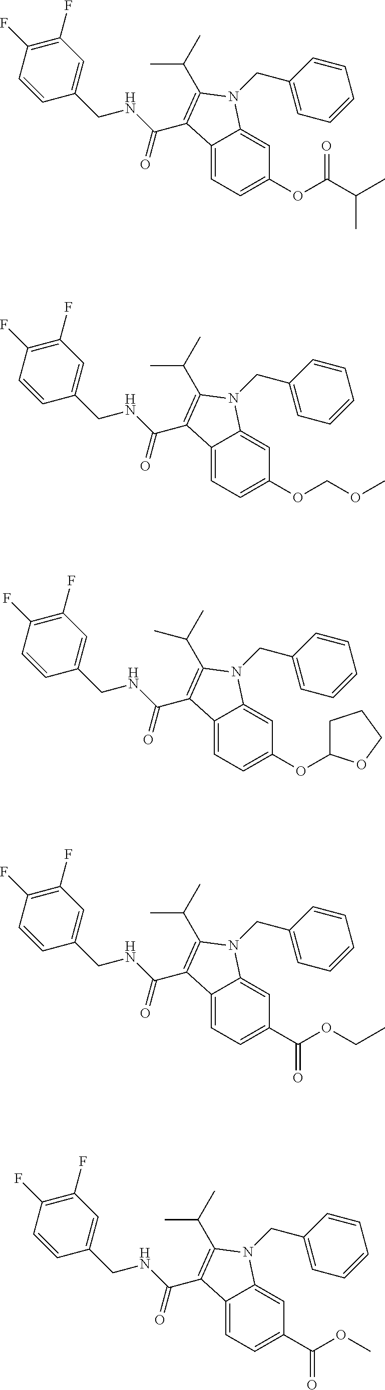

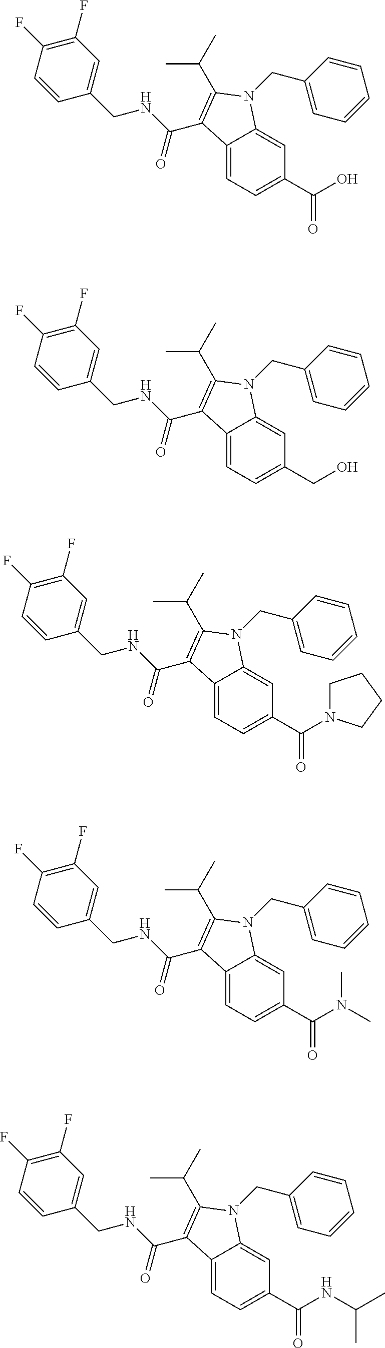

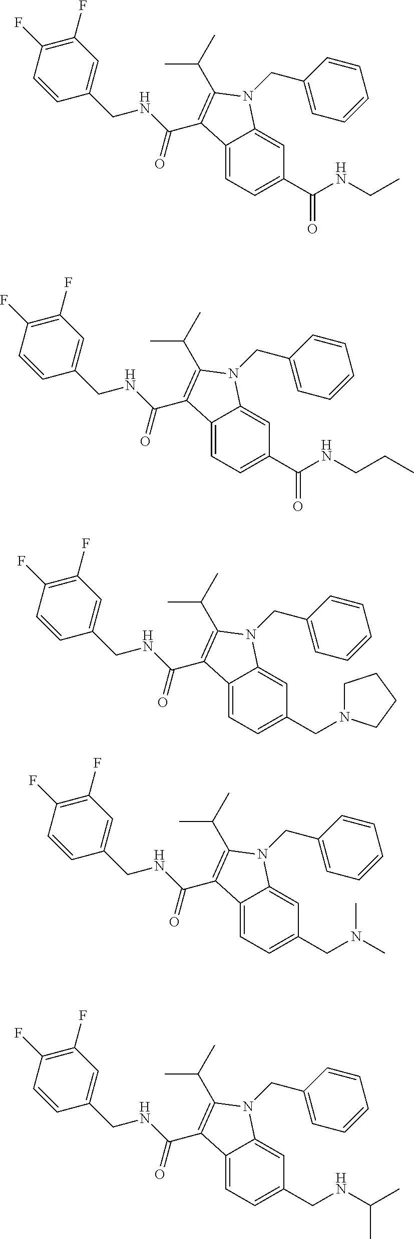

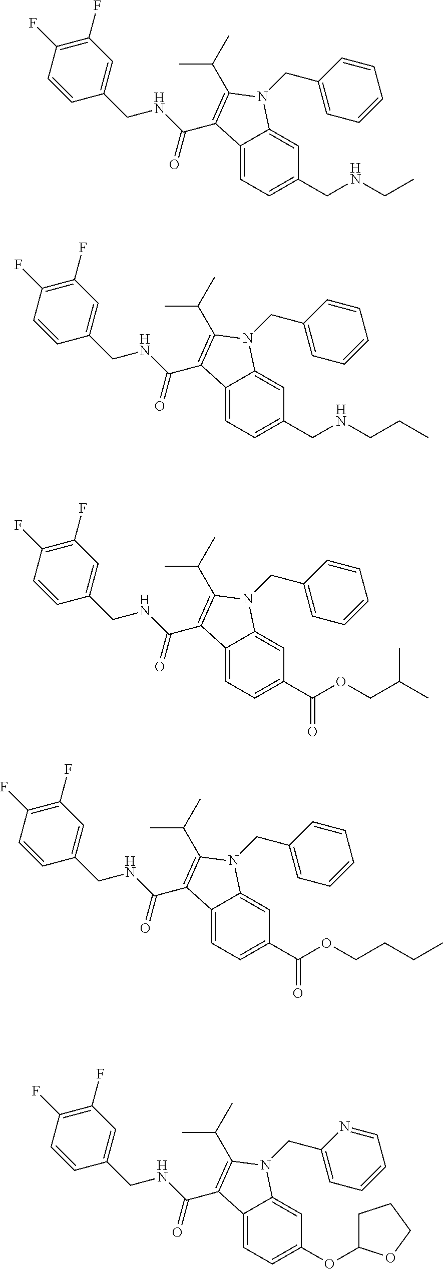

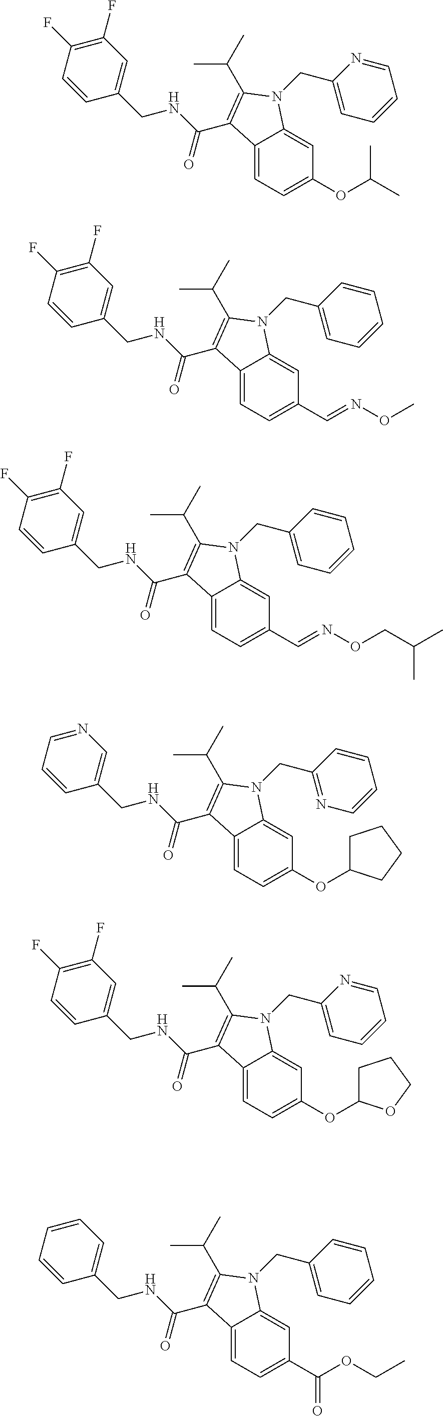

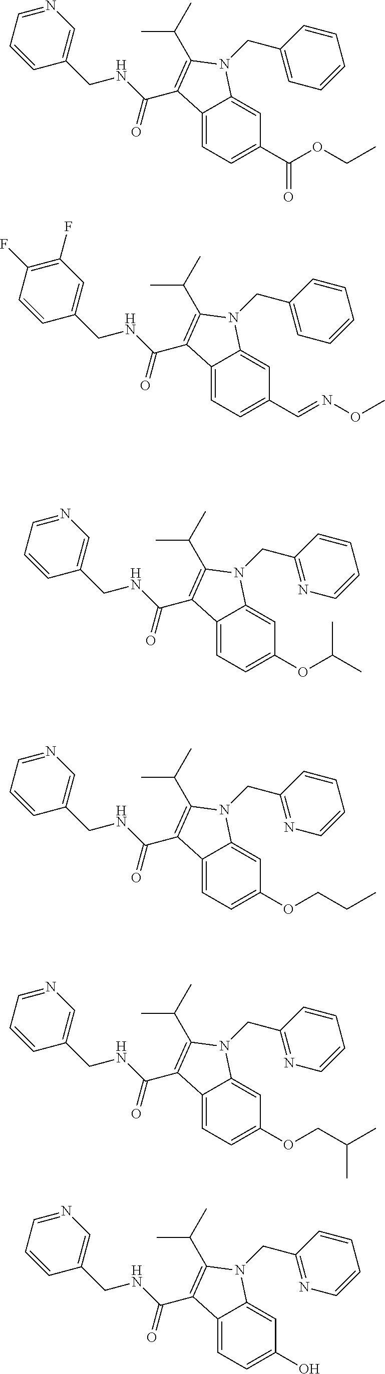

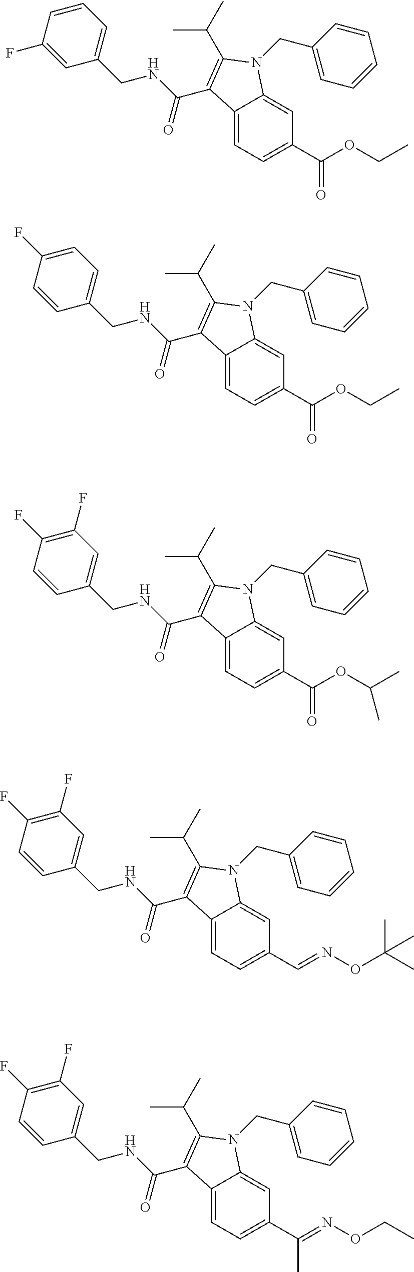

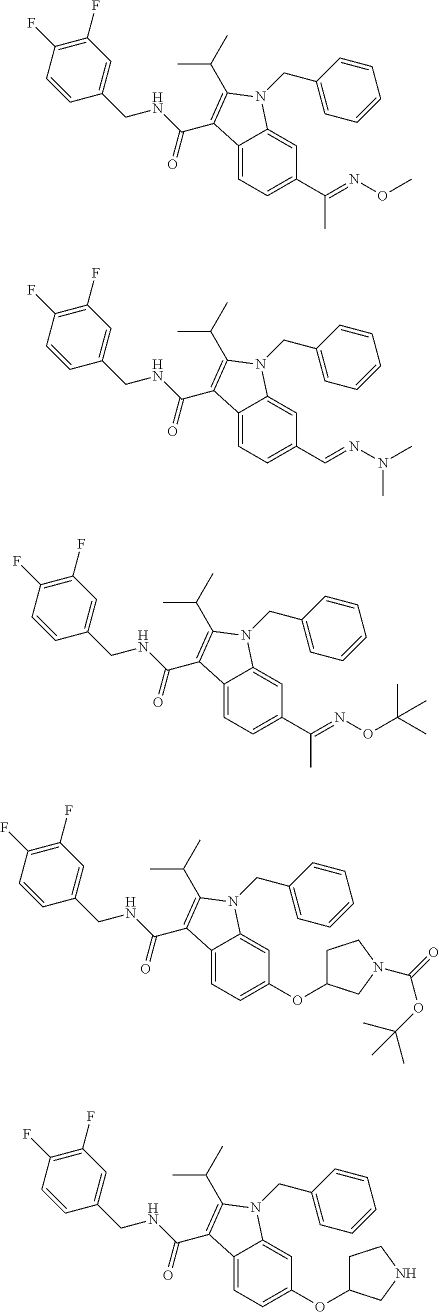

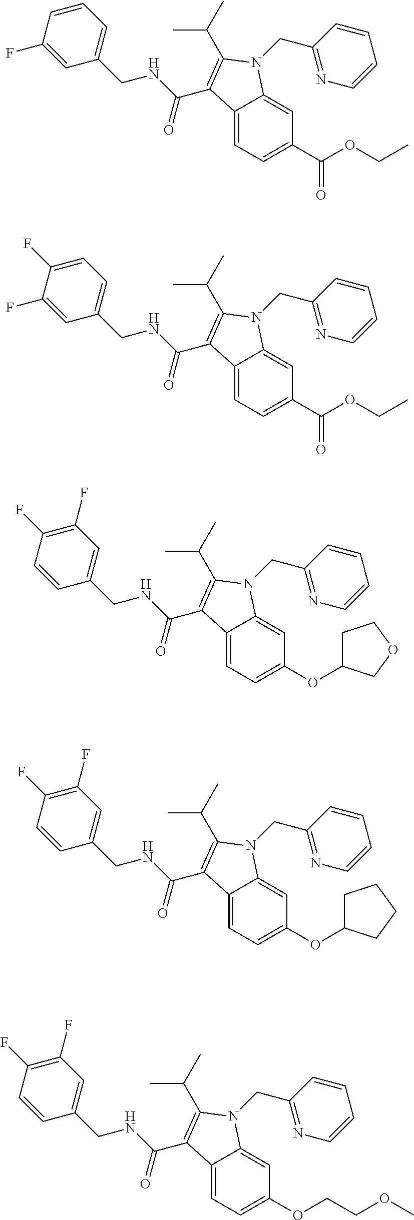

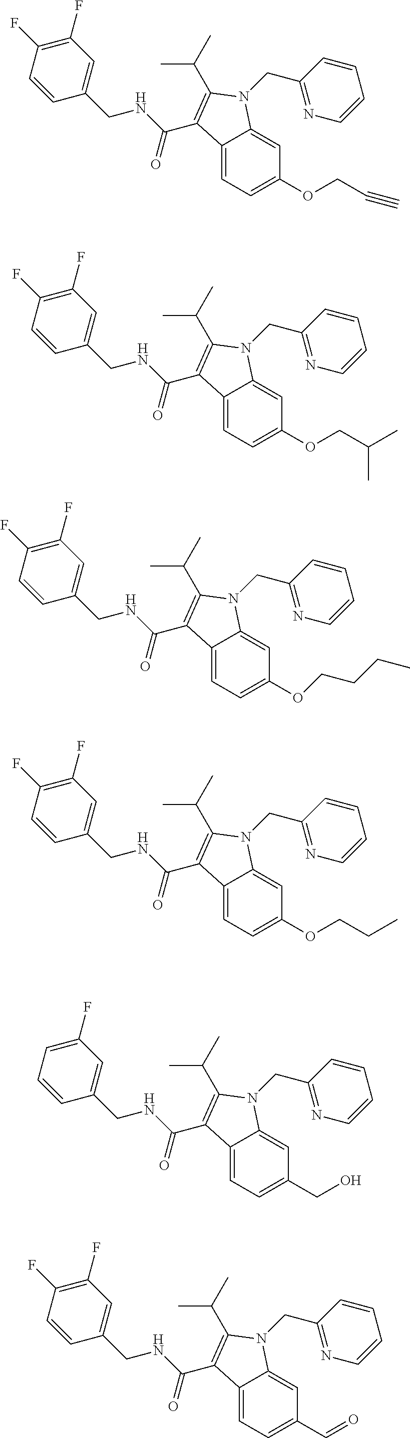

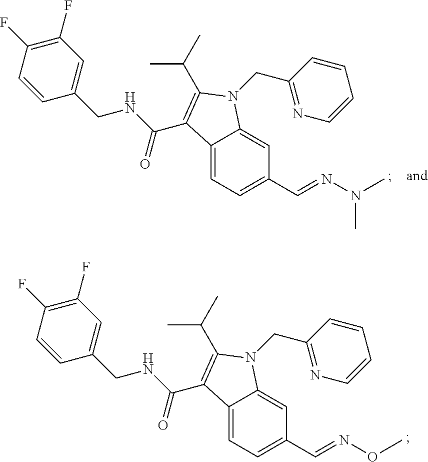

or a pharmaceutically acceptable salt thereof.

In another embodiment, the present invention provides a method for treating conditions of the eye, the method comprising the step of administering to a patient in need of such treatment a compound represented by the general formula III

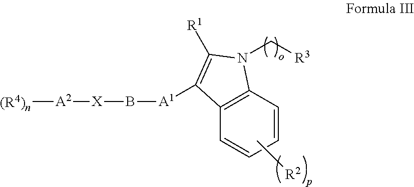

or a pharmaceutically acceptable salt thereof, wherein:

A¹ and A² are independently selected from the group consisting of (CH₂)m where m is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

B is selected from the group consisting of (CH₂)ₙ, where n is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, C═C(R⁵)₂, C═O, C═S, R⁵C═NR⁵, R⁵C═CR⁵, C═NOR⁵, CR⁵OR⁵, C(OR⁵)₂, CR⁵N(R⁵)₂, C(N(R⁵)₂)₂, CR⁵SR⁵, C(SR⁵)₂, SO, SO₂, and heterocyclic aryl comprising from 2 to 14 carbon atoms and from 1 to 3 heteroatoms selected from the group consisting of nitrogen, oxygen and sulfur;

X is selected from the group consisting of (CH₂)ᵣ, where r is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

provided that when m is 0 and B is C═O then X is not NR⁵, O or S;

Y is R⁶, or a carbocyclic aryl group comprising from 6 to 14 carbon atoms or a heterocyclic aryl group comprising from 2 to 14 carbon atoms and from 1 to 3 heteroatoms selected from the group consisting of nitrogen, oxygen and sulfur;

o is 0 or an integer of from 1 to 3;

p is 0 or an integer of from 1 to 4;

R¹, R², R³, R⁴ are independently selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxy, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, sulfonyl,

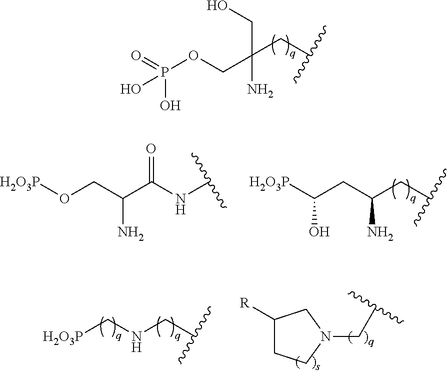

wherein R is CO₂H or PO₃H₂ and q is 0 or an integer of 1 to 5 and s is 0 or an integer from 1 to 3;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl; and

R⁶ is selected from the group consisting of straight or branched chain alkyl having 1 to 12 carbons, cycloalkyl having 3 to 6 carbons, alkenyl having 2 to 6 carbons and 1 or 2 double bonds and alkynyl having 2 to 6 carbons and 1 or 2 triple bonds.

In another embodiment, in Formula III, said aryl group is selected from the group consisting of benzene, pyridine, pyrazine, pyridazine, pyrimidine, triazine, thiophene, furan, thiazole, thiadiazole, isothiazole, oxazole, oxadiazole, isooxazole, naphthalene, quinoline, tetralin, chroman, thiochroman, tetrahydroquinoline, dihydronaphthalene, tetrahydronaphthalene, chromene, thiochromene, dihydroquinoline, indan, dihydrobenzofuran, dihydrobenzothiophene, indene, benzofuran, benzothiophene, coumarin and coumarinone, which aryl is unsubstituted or is substituted with one or two alkyl, alkenyl, alkynyl, aryl, halo, haloalkyl, hydroxyl, alkoxyl, alkylcarbonyl, formyl, oxycarbonyl, carboxyl, alkyl carboxylate, alkyl amide, aminocarbonyl, amino, cyano, diazo, nitro, thio, sulfoxyl, or sulfonyl groups.

In another embodiment, in Formula IIII, o is 1 and R³ is phenyl.

In another embodiment, in Formula IIII, R¹ is i-propyl.

In another embodiment, in Formula IIII, p is 1 and R² is hydroxy methyloxymethyloxy or dihydropyranyloxy.

In another embodiment, in Formula IIII, B is selected from the group consisting of C═C(R⁵)₂, C═O and C═NOR⁵.

In another embodiment, in Formula IIII, Y is R⁶.

In another embodiment, in Formula IIII, Y is R⁶ which is selected from the group consisting of methyl, n-propyl, and i-butyl.

In another embodiment, in Formula IIII, Y is selected from the group consisting of phenyl and 2,5 difluoro phenyl.

In another embodiment, in Formula IIII, p is 0 or p is 1 and R² is selected from the group consisting of hydroxy and dihyropyranyloxy.

In another embodiment, in Formula IIII, A¹ and A² are absent, B is C═O and X is ethyl or ethenyl.

In another embodiment, in Formula IIII, A¹ and A² are absent, B is C₂H₄ and X is CH₂.

In another embodiment, in Formula IIII, A¹ and A² are absent, B is sulfonyl; and X is NH.

In another embodiment, in Formula IIII, A¹, A² and B are absent and X is oxadiazolyl.

In another embodiment, in Formula IIII, A¹ is absent, B is C═O, X is NH and A² is NH.

In another embodiment, the compound of Formula III is selected from the group consisting of
1-Benzyl-3-((3,5-difluorobenzylamino)methyl)-2-isopropyl-1H-indol-5-ol,
(E)-1-Benzyl-5-hydroxy-2-isopropyl-1H-indole-3-carboxaldehyde, O-Benzyl Oxime,
(E)-1-Benzyl-5-hydroxy-2-isopropyl-1H-indole-3-carbaldehyde, O-Phenyl Oxime,
(E)-1-(1-Benzyl-5-hydroxy-2-isopropyl-1H-indol-3-yl)-3-phenylpropenone,
1-(1-Benzyl-5-hydroxy-2-isopropyl-1H-indol-3-yl)-3-phenylpropan-1-one,
1-(1-Benzyl-5-hydroxy-2-isopropyl-1H-indol-3-yl)ethanone,
1-(1-Benzyl-5-hydroxy-2-isopropyl-1H-indol-3-yl)butan-1-one,
1-(1-Benzyl-5-hydroxy-2-isopropyl-1H-indol-3-yl)-3-methylbutan-1-one,
1-(1-Benzyl-5-hydroxy-2-isopropyl-1H-indol-3-yl)-2-phenylethan-1-one,
(E)-1-(1-Benzyl-5-hydroxy-2-isopropyl-1H-indol-3-yl)-3-(3,4-difluorophenyl)prop-2-en-1-one and
1-(1-Benzyl-5-hydroxy-2-isopropyl-1H-indol-3-yl)-3-(3,4-difluorophenyl)propan-1-one.

In another embodiment, the present invention provides a method of treating conditions of the eye, the method comprising the step of administering to a patient in need of such treatment a compound represented by the general formula IV

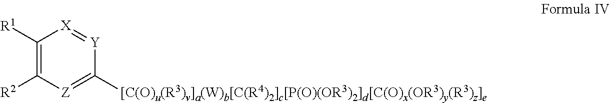

or a pharmaceutically acceptable salt thereof, wherein

X is selected from the group consisting of CR³ and N;

Y is selected from the group consisting of CR³ and N;

Z is selected from the group consisting of CR³ and N;

at least one of X, Y and Z is N;

W is NR³ or O;

R¹ is an aryl group;

R² is an aryl group;

R³ is selected from the group consisting of H and alkyl; and 2 of said R³ groups may together with N may form a heterocylic ring having from 2 to 6 carbon atoms;

R⁴ is selected from the group consisting of H, alkyl, OR³, and N(R³)₂;

a is 0 or an integer of from 1 to 6;

b is 0 or 1;

c is 0 or an integer of from 1 to 6;

d is 0 or 1;

e is 0 or 1;

u is 0 or 1;

v is 0 or an integer of from 1 to 2;

x is 0 or 1;

y is 0 or an integer of from 1 to 3;

z is 0 or an integer of from 1 to 3;

provided, however, that when d is 0, e is 1, and when e is 0, d is 1.

In another embodiment, in Formula IV:

R¹ is selected from the group consisting of phenyl and substituted derivatives thereof;

R² is selected from the group consisting of phenyl, furanyl, thienyl, pyridyl, pyranyl and substituted derivatives thereof;

R³ is selected from the group consisting of H and lower alkyl;

R⁴ is selected from the group consisting of H and lower alkyl;

a is 0 or an integer of from 1 to 3; and

c is 0 or an integer of from 1 to 5;

In another embodiment, in Formula IV, e is 0.

In another embodiment, in Formula IV, R¹ is represented by the general formula

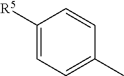

wherein R⁵ is selected from the group consisting of H, alkyl, trifluoromethyl, trifluoromethyloxy, halo and lower alkylthio.

In another embodiment, in Formula IV, R² is selected from the group consisting of furanyl, thienyl, pyridyl and pyranyl or R² is represented by the general formula

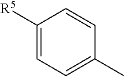

wherein R⁵ is selected from the group consisting of H, alkyl, trifluoromethyl, trifluoromethyloxy, halo, and lower alkylthio.

In another embodiment, in Formula IV, R³ is H.

In another embodiment, in Formula IV, wherein c is 1, 2 or 3.

In another embodiment, in Formula IV, a is 1.

In another embodiment, in Formula IV, wherein Z is N and X and Y are CR³.

In another embodiment, in Formula IV, W is NR³, R² is phenyl and R⁵ is selected from the group consisting of H and methyl.

In another embodiment, in Formula IV, R² is pyridyl and R⁵ is ethyl, and W is NR³.

In another embodiment, in Formula IV, d is 0.

In another embodiment, in Formula IV, R^{al} is represented by the general formula

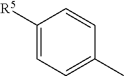

wherein R⁵ is selected from the group consisting of H, alkyl, trifluoromethyl, trifluoromethyloxy, halo, and loweralkylthio

In another embodiment, in Formula IV, R² is represented by the general formula

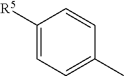

wherein R⁵ is selected from the group consisting of H, lower alkyl, trifluoromethyl, trifluoromethyloxy, halo, and lower alkylthio or R² is selected from the group consisting of furanyl, thienyl, pyridyl and pyranyl.

In another embodiment, in Formula IV, R³ is H.

In another embodiment, in Formula IV, a is 1.

In another embodiment, in Formula IV, x is 1 and z is 0.

In another embodiment, in Formula IV, R⁴ is selected from the group consisting of H, methyl, and ethyl.

In another embodiment, in Formula IV, Z is N, X and Y are CR³, R² is pyridyl, and R⁵ is selected from the group consisting of H, methyl, ethyl, propyl and trifluoromethyl.

In another embodiment, in Formula IV, X, Y and Z are N, R⁵ is selected from the group consisting of H, methyl, ethyl, propyl and trifluoromethyl.

In another embodiment, in Formula IV, X and Z are N and Y is CR³.

In another embodiment, in Formula IV, y is 0.

In another embodiment, the compound of Formula IV is selected from the group consisting of

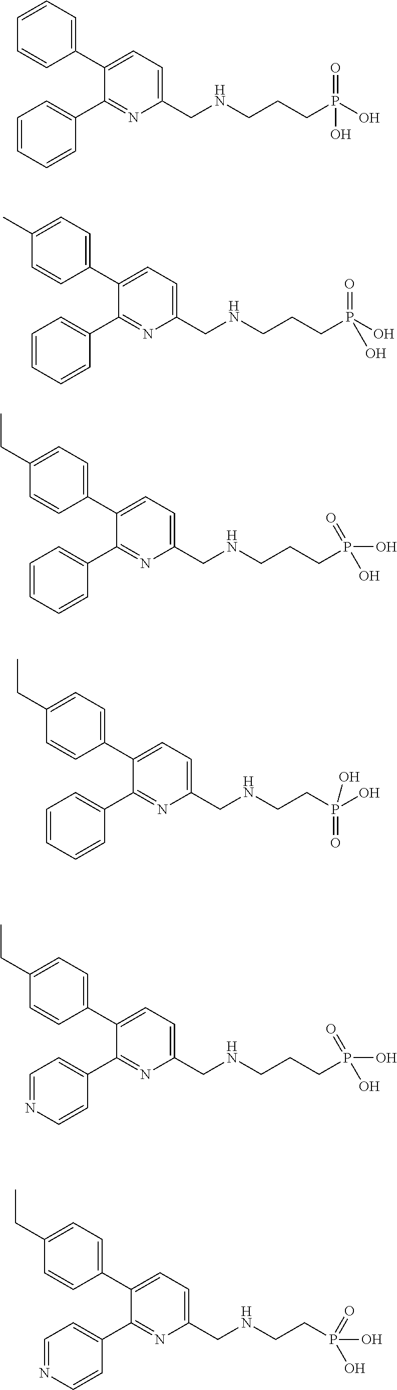

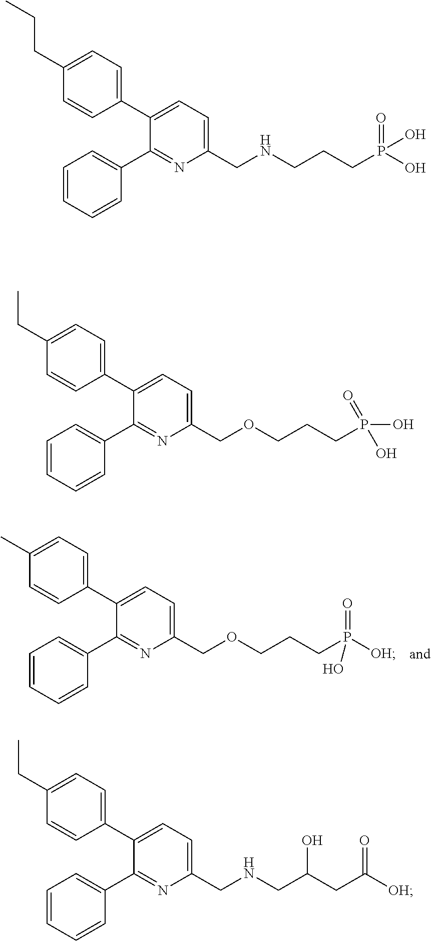

or a pharmaceutically acceptable salt thereof.

In another embodiment, the compound of Formula IV is selected from the group consisting of

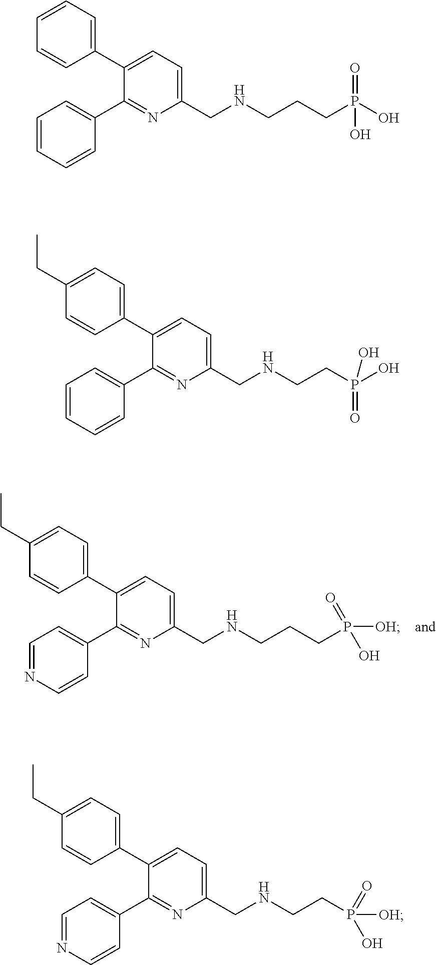

or a pharmaceutically acceptable salt thereof.

In another embodiment, the present invention provides a method of treating conditions of the eye, the method comprising the step of administering to a patient in need of such treatment an S1P3 receptor inhibitor comprising a 6-membered heteroaromatic ring including one, two or three enchained nitrogen atoms and the remaining ring atoms being carbon, an aryl radical directly bonded to said 6-membered heteroaromatic ring at both of the 5 and 6 positions and a side chain at the 2 position of said 6-membered heteroaromatic ring, wherein said side chain terminates with an end group selected from the group consisting of a phosphonic acid, a lower alkyl ester thereof, a carboxylic acid, a lower alkyl ester thereof, a lower alkyl ether and a lower alkylcarboxy, and any pharmaceutically acceptable salt thereof.

In another embodiment, in the above-mentioned heteroaromatic ring, the one, two or three enchained nitrogen atoms are at the 1, or 1 and 3, or 1 and 4, or 1, 3 and 4 positions, respectively.

In another embodiment, the present invention also provides a method for treating conditions of the eye, the method comprising administering to a patient in need of such treatment a compound represented by the general formula V:

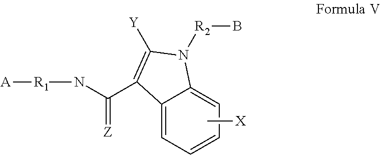

or a pharmaceutically acceptable salt thereof, wherein:

R₁ and R₂ are each independently (CH₂)ₙ, wherein n is an integer from 1 to 4;

A and B are each independently an aryl ring having 0, 1, 2, or 3 substituents consisting of from 0 to 8 carbon atoms, 0 to 3 oxygen atoms, 0 to 3 halogen atoms, 0 to 2 nitrogen atoms, 0 to 2 sulfur atoms, and from 0 to 24 hydrogen atoms;

X and Y are each independently H, alkyl of 1 to 8 carbons, or hydroxyalkyl of 1 to 8 carbons; and

Z is O or S.

In another embodiment, the compound of Formula V is represented by the general formula V-A

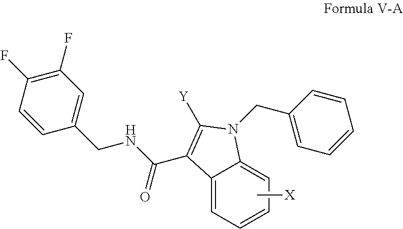

wherein X and Y are each independently H, unsubstituted alkyl of 1 to 4 carbons, hydroxyl, or unsubstituted alkoxy of 1 to 4 carbons.

In another embodiment, the compound of Formula V is selected from the group consisting of
1-benzyl-N-(3,4-difluorobenzyl)-2-isopropyl-6-propoxy-1H-indole-3-carboxamide,
1-benzyl-N-(3,4-difluorobenzyl)-6-isopropoxy-2-isopropyl-1H-indole-3-carboxamide,
1-benzyl-N-(3,4-difluorobenzyl)-5-hydroxy-2-isopropyl-1H-indole-3-carboxamide,
1-benzyl-2-cyclopentyl-N-(3,4-difluorobenzyl)-5-hydroxy-1H-indole-3-carboxamide,
1-benzyl-N-(3,4-difluorobenzyl)-6-ethoxy-2-isopropyl-1H-indole-3-carboxamide,
1-benzyl-N-(3,4-difluorobenzyl)-2-isopropyl-1H-indole-3-carboxamide, and
2-cyclopentyl-N-(3,4-difluorobenzyl)-5-hydroxy-1-(pyridin-2-ylmethyl)-1H-indole-3-carboxamide; or a pharmaceutically acceptable salt thereof.

In another embodiment, the present invention provides a method for treating conditions of the eye, the method comprising administering to a patient in need of such treatment a compound represented by the general formula VI

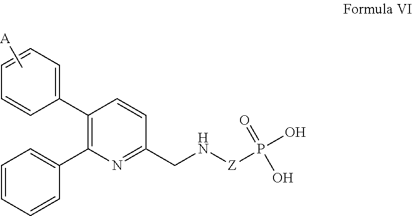

or a pharmaceutically acceptable salt thereof, wherein A is a phenyl ring having 0, 1, 2, or 3 substituents consisting of from 0 to 6 carbon atoms and from 0 to 13 hydrogen atoms; and
Z is (CH₂)ₙ, wherein n is an integer from 1 to 4.

In another embodiment, the compound of Formula VI is 3-((5-(4-ethylphenyl)-6-phenylpyridin-2-yl)methylamino)propylphosphonic acid.

In another embodiment, the S1P3 receptor inhibitor of the present invention is selective for the S1P3 receptor as compared to one or more receptors selected from the group consisting of the S1P1 receptor, S1P2 receptor, S1P4 receptor, and S1P5 receptor.

In another embodiment, in Formula IV, the condition of the eye sought to be treated by the compounds of present invention is selected from the group consisting of conditions affecting the posterior part of the eye, such as maculopathies and retinal degeneration including non-exudative age related macular degeneration, exudative age related macular degeneration, choroidal neovascularization, diabetic retinopathy, acute macular neuroretinopathy, central serous chorioretinopathy, cystoid macular edema, and diabetic macular edema; uveitis, retinitis, and choroiditis such as acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; vasuclar diseases/exudative diseases such as retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, and bone marrow transplant retinopathy; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigement epitheliitis.

### DETAILED DESCRIPTION OF THE INVENTION

### S1P3 Receptor

Sphingosine-1-phosphate (“S1P”) is an important chemical messenger that can activate particular cell surface transmembrane G-protein coupled receptors known as endothelial gene differentiation (“Edg”) receptors.

There are five known S1P receptors activated by S1P: S1P1, also known as Edg 1 (human Edg-1, GenBank Accession No. AF233365); S1P2, also known as Edg 5 (human Edg-5, GenBank Accession No. AF034780); S1P3, also known as Edg 3 (human Edg-3, GenBank Accession No. X83864); S1P4, also known as Edg 6 (human Edg-6, GenBank Accession No. AF000479); and S1P5, also known as Edg 8 (human Edg-8, GenBank Accession No. AF317676).

The method of the present invention treats conditions of the eye by administering compounds that inhibit the S1P3 receptor. In one embodiment, the method administers compounds that selectively inhibit the S1P3 subtype as compared to at least one other S1P subtype.

### S1P3 Receptor Inhibitors

A compound is an “S1P3 receptor inhibitor” if it inhibits, partially or completely, the cellular response caused by binding of S1P or other ligand to the S1P3 receptor. S1P3 is a G-protein coupled receptor (GPCR). When a ligand binds to that receptor it induces a conformational shift, causing GDP to be replaced by GTP on the α-subunit of the associated G-proteins and subsequent release of the G-proteins into the cytoplasm. The α-subunit then dissociates from the βγ-subunit and each subunit can then associate with effector proteins, activating second messengers, and leading to a cellular response. The process is referred to as S1P cell signaling.

One example of a cellular response is the accumulation of cAMP. The effect of an inhibitor on this response can be measured by well-known techniques in the art. One example is radioimmunoassay and the [γ-³⁵S]GTP binding assay, illustrated in U.S. Patent Application Publication No. 2005/0222422 and No. 2007/0088002 to assay S1P agonists (the disclosures of both these publications are incorporated by reference). To evaluate a compound for its potential as an inhibitor, one can measure cAMP accumulation by radioimmunoassay after incubating S1P (or S1P receptor agonist) in the presence of a test compound and cells expressing the S1P3 receptor; if the compound is an inhibitor, it will reduce the activation of S1P3 by S1P, which can be measured as reduced cAMP accumulation.

Another method of determining if a compound is an S1P3 receptor inhibitor is with a FLIPR assay. An example of this method is described in U.S. patent application Ser. No. 11/675,168, the contents of which are incorporated herein by reference. According to that application, compounds may be assessed for their ability to activate or block activation of the human S1P3 receptor in T24 cells stably expressing the human S1P3 receptor. In this assay ten thousand cells/well are plated into 384-well poly-D-lysine coated plates one day prior to use. The growth media for the S1P3 receptor expressing cell line is McCoy's 5A medium supplemented with 10% charcoal-treated fetal bovine serum (FBS), 1% antibiotic-antimycotic and 400 μg/ml geneticin. On the day of the experiment, the cells are washed twice with Hank's Balanced Salt Solution supplemented with 20 mM HEPES (HBSS/Hepes buffer). The cells are then dye loaded with 2 uM Fluo-4 diluted in the HBSS/Hepes buffer with 1.25 mM Probenecid and incubated at 37° C. for 40 minutes. Extracellular dye is removed by washing the cell plates four times prior to placing the plates in the FLIPR (Fluorometric Imaging Plate Reader, Molecular Devices). Ligands are diluted in HBSS/Hepes buffer and prepared in 384-well microplates. The positive control, S1P, is diluted in HBSS/Hepes buffer with 4 mg/ml fatty acid free bovine serum albumin. The FLIPR transfers 12.5 μl from the ligand microplate to the cell plate and takes fluorescent measurements for 75 seconds, taking readings every second, and then for 2.5 minutes, taking readings every 10 seconds. Drugs are tested over the concentration range of 0.61 nM to 10,000 nM. Data for Ca⁺² responses is obtained in arbitrary fluorescence units and not translated into Ca⁺² concentrations. IC₅₀ values are determined through a linear regression analysis using the Levenburg Marquardt algorithm.

S1P3 receptor inhibitors include S1P3 receptor antagonists and S1P3 receptor inverse agonists, as long as they inhibit, partially or completely, S1P cell signaling.

S1P3 receptor inhibitors may be selective for the S1P3 receptor or they may inhibit S1P cell signaling at more than one of the S1P receptor subtypes. An inhibitor is selective for the S1P3 receptor compared to another S1P subtype if the inhibitor is more than 100 times as potent at inhibiting the S1P3 receptor than it is at inhibiting or activating the other S1P receptor subtype. For example, the IC₅₀ of hypothetical compound A in a FLIPR assay is 100 nM at the S1P3 receptor, >5000 nM at the S1P1 receptor, and 200 nM at the S1P5 receptor; compound A is selective for the S1P3 receptor compared to the S1P1 receptor but not compared to the S1P5 receptor. If, to take another example, the IC₅₀ of hypothetical compound B is 100 nM at the S1P3 receptor and EC₅₀ is 200 nM at the S1P1 receptor and >5000 at the S1P2 receptor, then compound B is selective for the S1P3 receptor compared to the S1P2 receptor but not the S1P1 receptor.

In one embodiment, the S1P3 receptor inhibitors are selective for the S1P3 receptor as compared to one receptor selected from the group consisting of the S1P1, S1P2, S1P4, and S1P5 receptors. In another embodiment, the S1P3 receptor inhibitors are selective for the S1P3 receptor as compared to two receptors selected from the group consisting of the S1P1, S1P2, S1P4, and S1P5 receptors. In another embodiment, the S1P3 receptor inhibitors are selective for the S1P3 receptor as compared to three receptors selected from the group consisting of the S1P1, S1P2, S1P4, and S1P5 receptors. In another embodiment, the S1P3 receptor inhibitors are selective for the S1P3 receptor as compared to S1P1, S1P2, S1P4, and S1P5 receptors.

### S1P3 Receptor Inhibitors Useful in the Method of the Invention

In one embodiment, S1P3 receptor inhibitors useful in the method of the invention include anti-S1P3 receptor antibodies, such as polyclonal, monoclonal, humanized, bispecific, and heteroconjugate antibodies. In another embodiment, S1P3 receptor inhibitors useful in the method of the invention include small molecule inhibitors.

### Polyclonal Antibodies

Polyclonal antibodies may be raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R₁N═C═NR, where R and R₁ are different alkyl groups.

Animals can be immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

### Monoclonal Antibodies

Monoclonal antibodies may be made using the hybridoma method first described by Kohler et al., Nature, 256:495 (1975), or may be made by recombinant DNA methods (U.S. Pat. No. 4,816,567, the disclosure of which is incorporated herein by refernece).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs. 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567, the disclosure of which is incorporated herein by reference; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Human and Humanized Antibodies

The anti-S1P3 receptor antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-S1P3 receptor antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J.sub.H) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669; U.S. Pat. No. 5,545,807; and WO 97/17852 (the disclosures of the foregoing patent references are incorporated by reference herein).

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275, incorporated herein by refernece).

### Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′).sub.2 fragments (Carter et al., Bio/Technology 10: 163-167 (1992)). According to another approach, F(ab′).sub.2 fragments can be isolated directly from recombinant host cell culture. Fab and F(ab′).sub.2 fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458, the disclosures of which are incorporated by reference. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example, the disclosure of which is incorporated by reference. Such linear antibody fragments may be monospecific or bispecific.

### Bispecific Antibodies

Bispecific antibodies are antibodies that have binding specificities for at least two different epitopes. Exemplary bispecific antibodies may bind to two different epitopes of an S1P3 receptor described herein. Other such antibodies may combine an S1P3 receptor binding site with a binding site for another polypeptide. Alternatively, an anti-51P3 receptor antibody arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the S1P3 receptor-expressing and/or binding cell. Bispecific antibodies may also be used to localize cytotoxic agents to cells which express and/or bind the S1P3 receptor. These antibodies possess a S1P3 receptor binding arm and an arm which binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcγ antibody is shown in WO98/02463. U.S. Pat. No. 5,821,337 teaches a bispecific anti-ErbB2/anti-CD3 antibody. The disclosures of all of these references are incorporated herein by reference.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, the disclosure of which is incorporated by reference, and in Traunecker et al., EMBO J. 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168 (incorporated herein by reference), the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147:60 (1991).

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1).sub.n-VD2-(X2).sub.n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH 1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### Small Molecule Inhibitors

In one embodiment of the invention, S1P3 receptor inhibitors useful in the method of the invention include those disclosed in U.S. patent application Ser. No. 11/675,168, Ser. No. 11/690,637, No. 60/884,470, and No. 60/824,807, and in U.S. Patent Application Publication No. 2005/0222422, No. 2007/0032459 and No. 2008/0025973. The disclosures of all the foregoing references are incorporated by reference.

### DEFINITIONS

In describing S1P3 receptor inhibitors useful in the invention, the following terms have the following meanings, unless otherwise indicated.

“Me” refers to methyl.

“Et” refers to ethyl.

“tBu” refers to t-butyl.

“iPr” refers to i-propyl.

“Ph” refers to phenyl.

“Alkyl” refers to a straight-chain, branched or cyclic saturated aliphatic hydrocarbon. The alkyl group may have 1 to 12 carbons; in other embodiments, it is a lower alkyl of from 1 to 7 carbons, or a lower alkyl from 1 to 4 carbons. Typical alkyl groups include methyl, ethyl, propyl, isopropyl, butyl, isobutyl, tertiary butyl, pentyl, hexyl and the like. The alkyl group may be optionally substituted with one or more substituents are selected from the group consisting of hydroxyl, cyano, alkoxy, =0, =S, NO₂, halogen, dimethyl amino and SH.

“Alkenyl” refers to a straight-chain, branched or cyclic unsaturated hydrocarbon group containing at least one carbon-carbon double bond. The alkenyl group may have 2 to 12 carbons; in other embodiments, it is a lower alkenyl of from 2 to 7 carbons, or a lower alkenyl of from 2 to 4 carbons. The alkenyl group may be optionally substituted with one or more substituents selected from the group consisting of hydroxyl, cyano, alkoxy, O, S, NO₂, halogen, dimethyl amino and SH.

“Alkynyl” refers to a straight-chain, branched or cyclic unsaturated hydrocarbon containing at least one carbon-carbon triple bond. The alkynyl group may have 2 to 12 carbons; in other embodiments, it is a lower alkynyl of from 2 to 7 carbons, or a lower alkynyl of from 2 to 4 carbons. The alkynyl group may be optionally substituted with one or more substituents selected from the group consisting of hydroxyl, cyano, alkoxy, O, S, NO₂, halogen, dimethyl amino and SH.

“Alkoxy” refers to an “O-alkyl” group.

“Aryl” refers to an aromatic group which has at least one ring having a conjugated pi electron system and includes carbocyclic aryl, heterocyclic aryl and biaryl groups. The aryl group may be optionally substituted with one or more substituents selected from the group consisting of halogen, trihalomethyl, hydroxyl, SH, OH, NO₂, amine, thioether, cyano, alkoxy, alkyl, and amino.

“Alkaryl” (Alkylaryl) refers to an alkyl that is covalently joined to an aryl group. In one embodiment, the alkyl is a lower alkyl.

“Aryloxy” refers to an “O-aryl” group.

“Arylalkyloxy” refers to an “O-alkaryl” (O-alkylaryl) group.

“Carbocyclic aryl” refers to an aryl group wherein the ring atoms are carbon.

“Heterocyclic aryl” refers to an aryl group having from 1 to 3 heteroatoms as ring atoms, the remainder of the ring atoms being carbon. Heteroatoms include oxygen, sulfur, and nitrogen.

“Hydrocarbyl” refers to a hydrocarbon radical having only carbon and hydrogen atoms. The hydrocarbyl radical may have from 1 to 20 carbon atoms, or from 1 to 12 carbon atoms, or from 1 to 7 carbon atoms.

“Substituted hydrocarbyl” refers to a hydrocarbyl radical wherein one or more, but not all, of the hydrogen and/or the carbon atoms are replaced by a halogen, nitrogen, oxygen, sulfur or phosphorus atom or a radical including a halogen, nitrogen, oxygen, sulfur or phosphorus atom, e.g. fluoro, chloro, cyano, nitro, hydroxyl, phosphate, thiol, etc.

“Amide” refers to —C(O)—NH—R′, wherein R′ is alkyl, aryl, alkylaryl or hydrogen.

“Ester” refers to —C(O)—O—R′, wherein R′ is alkyl, aryl or alkylaryl.

“Carboxy” refers to —C(O)—O—H

“Thioamide” refers to —C(S)—NH—R′, wherein R′ is alkyl, aryl, alkylaryl or hydrogen.

“Thiol ester” refers to —C(O)—S—R′, wherein R′ is alkyl, aryl, alkylaryl or hydrogen.

“Amine” refers to a—N(R″)R′″ group, wherein R″ and R′″ are independently selected from the group consisting of alkyl, aryl, and alkylaryl.

“Thioether” refers to —S—R″, wherein R″ is alkyl, aryl, or alkylaryl.

“Sulfonyl” refers to —S(O)₂—R″″, wherein R″″ is alkyl, aryl, C(CN)═C-aryl, CH₂CN, or alkyaryl.

“Sulfoxyl” refers to —S(O)—R″″, wherein R″″ is alkyl, alkenyl, alkynyl, aryl, or alkylaryl.

“Sulfonamidyl” refers to —S(O)—NR'(R″), wherein R′ and R″ are independently alkyl, alkenyl, alkynyl, aryl, or alkylaryl.

“Carbocyclic” refers to any ring, aromatic or non-aromatic, containing 1 to 12 carbon atoms.

“Heterocyclic” refers to any ring, aromatic or non-aromatic, containing 1 to 12 carbon atoms and 1 to 4 heteroatoms chosen from a group consisting of oxygen, nitrogen and sulfur.

### Indole-3-Carboxylic Acid Amide, Ester, Thioamide and Thiol Ester Compounds Bearing Aryl or Heteroaryl Groups

U.S. patent application Ser. No. 11/675,168 discloses S1P3 receptor antagonists having the following formula:

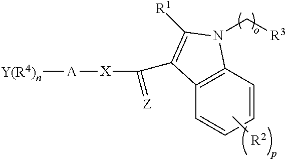

wherein

X is NR⁵, O, S;

Z is O or S;

n is 0 or an integer of from 1 to 4;

o is 0 or an integer of from 1 to 3;

p is 0 or an integer of from 1 to 4;

A is (C(R⁵)₂)ₘ, wherein
m is 0 or an integer of from 1 to 6;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl groups;

Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

R¹, R², R³, R⁴ are selected from the group consisting of hydrogen; straight or branched chain alkyl having 1 to 12 carbons; cycloalkyl having 3 to 6 carbons; alkenyl having 2 to 6 carbons and 1 or 2 double bonds; alkynyl having 2 to 6 carbons and 1 or 2 triple bonds; aryl wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur; halo; C₁ to C₁₂ haloalkyl; hydroxyl; C₁ to C₁₂ alkoxy; C₃ to C₂₀ arylalkyloxy; C₁ to C₁₂ alkylcarbonyl; formyl; oxycarbonyl; carboxy; C₁ to C₁₂ alkyl carboxylate; C₁ to C₁₂ alkyl amide; aminocarbonyl; amino; cyano; diazo; nitro; thio; sulfoxyl; sulfonyl groups; or a group selected from the group consisting of

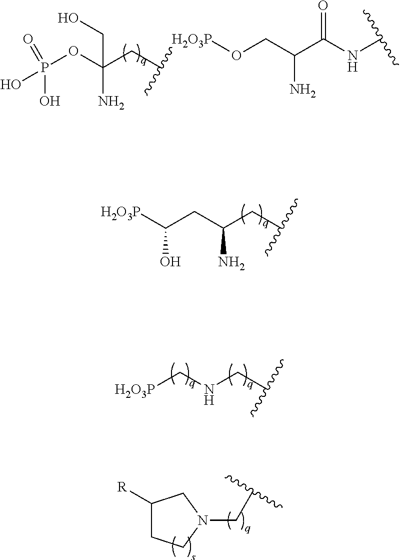

wherein R is CO₂H or PO₃H₂, p is an integer of 1 or 2 and q is 0 or an integer of 1 to 5 and s is 0 or an integer of 1 or 2; provided that, if Y is phenyl, it must be substituted with at least one R⁴ group that is not hydrogen.

Examples of such compounds include the followina

| NO. | COMPOUND |
|---|---|
| 1 | 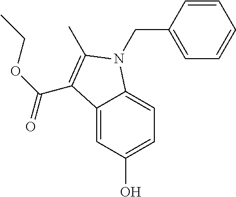 |
| 2 | 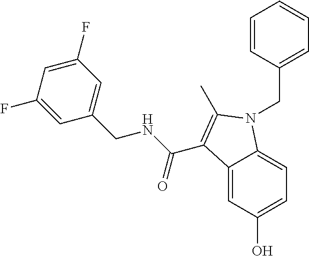 |
| 3 | 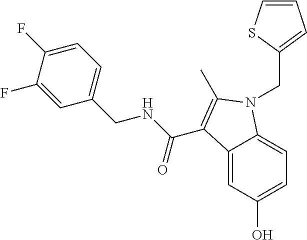 |
| 4 | 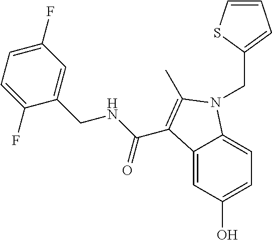 |
| 5 | 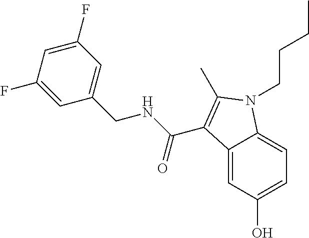 |
| 6 | 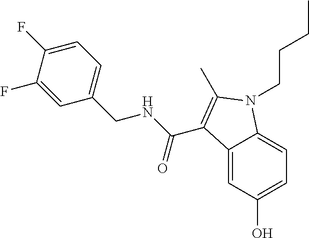 |
| 7 | 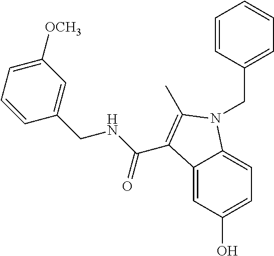 |
| 8 | 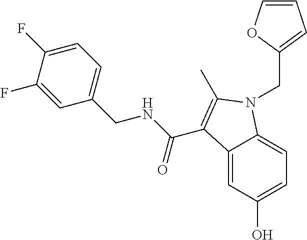 |
| 9 | 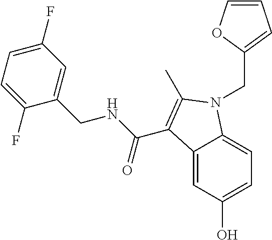 |
| 10 | 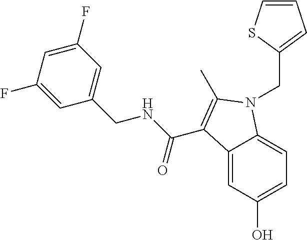 |
| 11 | 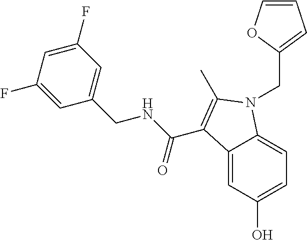 |
| 12 | 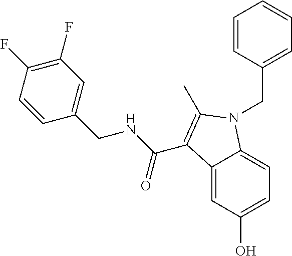 |
| 13 | 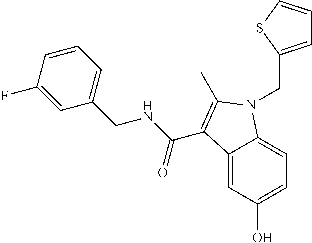 |
| 14 | 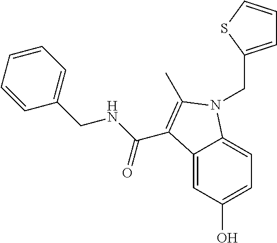 |
| 15 | 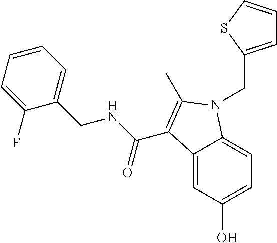 |
| 16 | 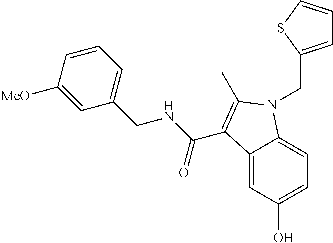 |
| 17 | 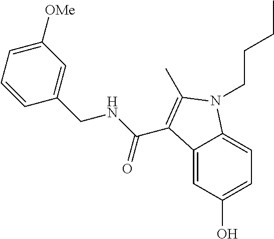 |
| 18 | 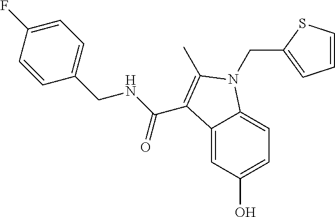 |
| 19 | 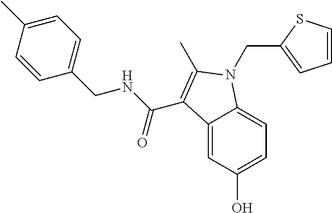 |
| 20 | 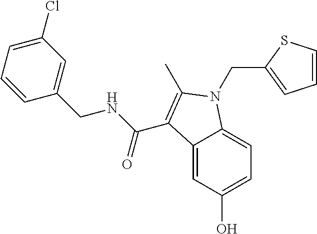 |
| 21 | 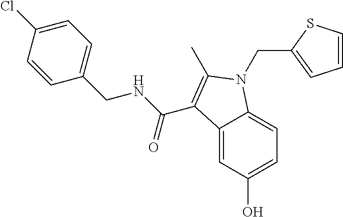 |
| 22 | 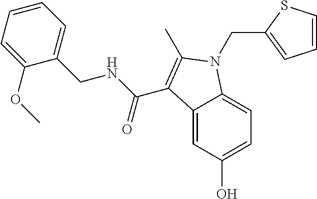 |
| 23 | 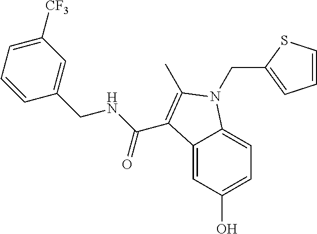 |
| 24 | 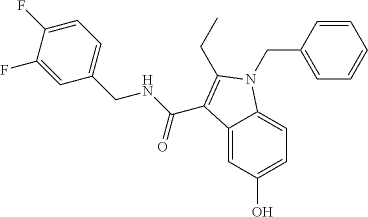 |
| 25 | 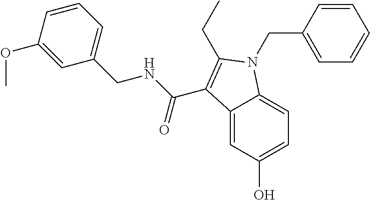 |
| 26 | 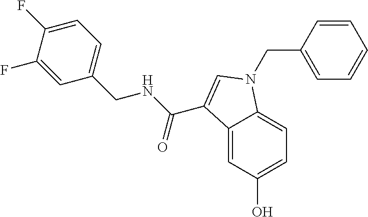 |
| 27 | 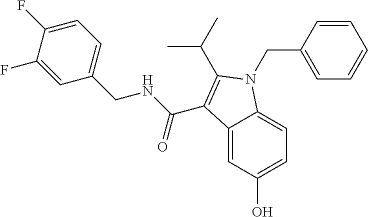 |
| 28 | 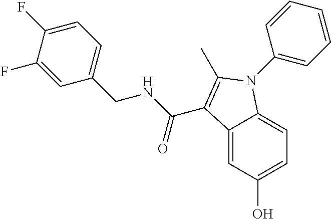 |
| 29 | 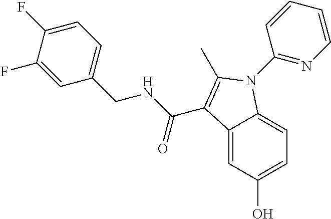 |
| 30 | 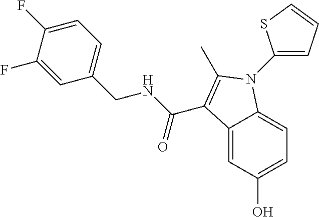 |
| 31 | 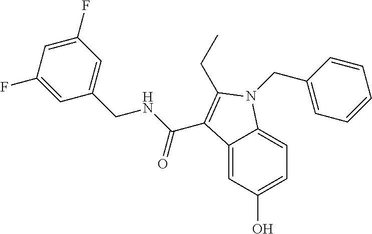 |
| 32 | 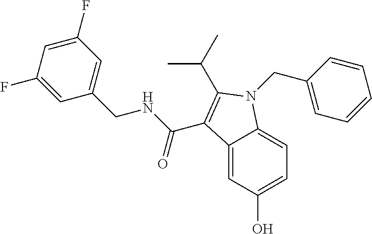 |
| 33 | 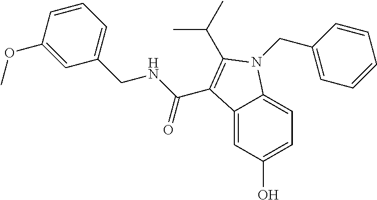 |
| 34 | 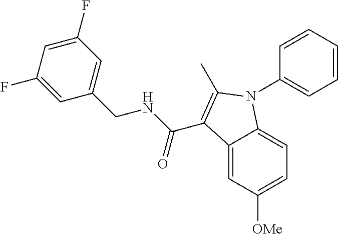 |
| 35 | 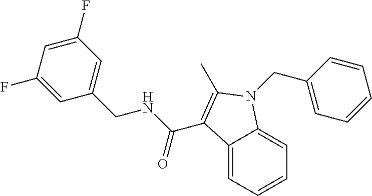 |
| 36 | 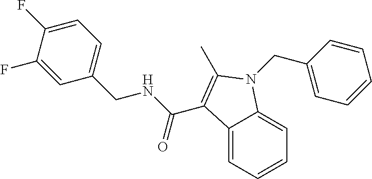 |
| 37 | 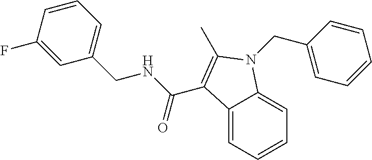 |
| 38 | 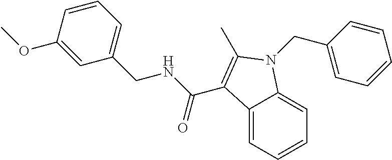 |
| 39 | 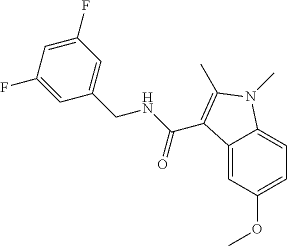 |
| 40 | 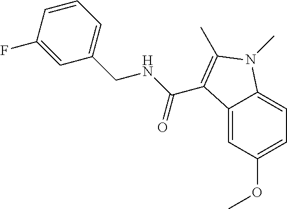 |
| 41 | 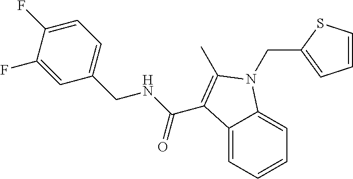 |
| 42 | 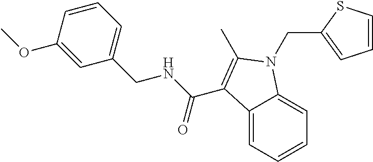 |
| 43 | 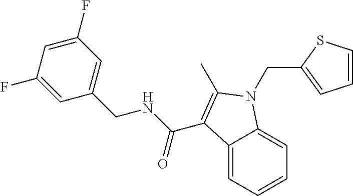 |
| 44 | 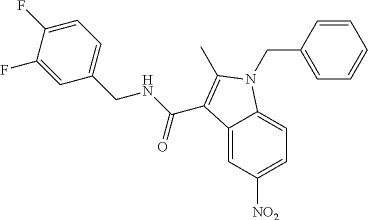 |
| 45 | 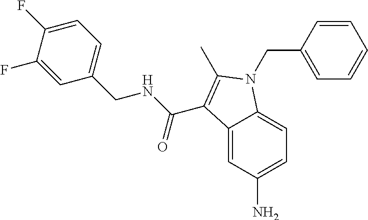 |
| 46 | 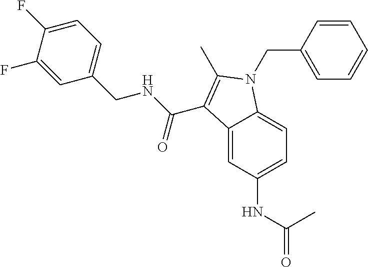 |
| 47 | 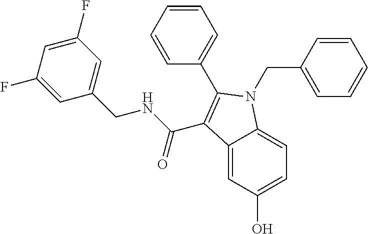 |
| 48 | 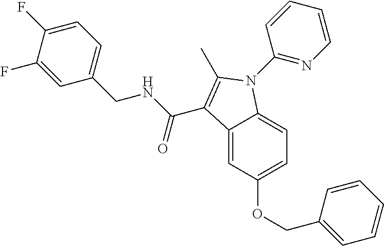 |

### Additional Indole Compounds

U.S. Patent Application No. 60/884,470 discloses S1P3 receptor antagonists having the following formula:

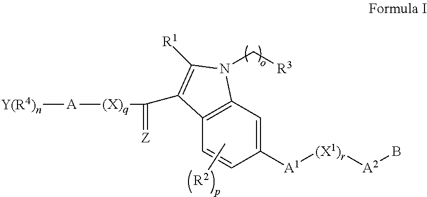

wherein:

R¹ R², R³ and R⁴ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, carbocyclic hydrocarbon groups having from 3 to 20 carbon atoms, heterocyclic groups having up to 20 carbon atoms and at least one of oxygen, nitrogen and/or sulfur in the ring, halo, C₁ to C₁₂ haloalkyl, hydroxyl, C₁ to C₁₂ alkoxy, C₃ to C₂₀ arylalkyloxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, and sulfonyl groups;

X and X¹ are independently selected from the group consisting of NR⁵, O and S;

R⁵ is hydrogen, an alkyl group of 1 to 10 carbons, a cycloalkyl group of 5 to 10 carbons, phenyl or lower alkylphenyl;

Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

Z is O or S;

n is 0 or an integer of from 1 to 5;

o is 0 or an integer of from 1 to 3;

p is 0 or an integer of from 1 to 3;

q is 0 or 1;

r is 0 or 1;

A, A¹ and A² are independently selected from the group consisting of (CH₂)ᵥ wherein v is 0 or an integer of from 1 to 12, branched chain alkyl having 3 to 12 carbons, cycloalkyl having 3 to 12 carbons, alkenyl having 2 to 10 carbons and 1-3 double bonds and alkynyl having 2 to 10 carbons and 1 to 3 triple bonds;

B is selected from the group consisting of hydrogen, OR⁶, COOR⁷, NR⁸R⁹, CONR⁸R⁹, COR¹⁰, CH═NOR¹¹, CH═NNR¹²R¹³ wherein R⁶, R⁷, R¹⁰ and R¹¹ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, R⁸, R⁹, R¹² and R¹³ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, or R⁸ and R⁹ and/or R¹² and R¹³, together, can form a divalent carbon radical of 2 to 5 carbons to form a heterocyclic ring with nitrogen, wherein any of R⁶, R⁷, R⁸, R⁹, R¹⁰, R¹¹, R¹² or R¹³ may be substituted with one or more halogen, hydroxy, alkyloxy, cyano, nitro, mercapto or thiol radical; provided however, when v is 0, and r is 0, B is not hydrogen; or B is a carbocyclic hydrocarbon group having from 3 to 20 carbon atoms, or a heterocyclic group having up to 20 carbon atoms and at least one of oxygen, nitrogen and/or sulfur in the ring, and wherein when said B is a carbocyclic or heterocyclic group B may be bonded to A² at any position, or a pharmaceutically acceptable salt of said compound.

The aryl group is a carbocyclic aryl or heterocyclic aryl group wherein said carbocylic aryl comprises from 6 to 20 atoms and said heterocyclic aryl comprise from 2 to 20 carbon atoms and from 1 to 5 heteroatoms selected from the group consisting of nitrogen, oxygen and sulfur, and preferably said aryl group is selected from the group consisting of benzene, pyridine, pyrazine, pyridazine, pyrimidine, triazine, thiophene, furan, thiazole, thiadiazole, isothiazole, oxazole, oxadiazole, isooxazole, naphthalene, quinoline, tetralin, chroman, thiochroman, tetrahydroquinoline, dihydronaphthalene, tetrahydronaphthalen, chromene, thiochromene, dihydroquinoline, indan, dihydrobenzofuran, dihydrobenzothiophene, indene, benzofuran, benzothiophene, coumarin and coumarinone. Said aryl groups can be bonded to the above moiety at any position. Said aryl group may itself be substituted with any common organic functional group including but not limited to C₁ to C₁₂ alkyl, C₂ to C₆ alkenyl, C₂ to C₆ alkynyl, halo, C₁ to C₁₂ haloalkyl, hydroxyl, C₁ to C₁₂ alkoxyl, C₁ to C₁₂ alkylcarbonyl, formyl, oxycarbonyl, carboxyl, C₁ to C₁₂ alkyl carboxylate, C₁ to C₁₂ alkyl amide, aminocarbonyl, amino, cyano, diazo, nitro, thio, sulfoxyl, or sulfonyl groups.

Preferably Z is O.

Preferably, the carbocyclic aryl group will comprise from 6 to 14 carbon atoms, e.g. from 6 to 10 carbon atoms. Preferably the heterocyclic aryl group will comprise from 2 to 14 carbon atoms and one or more, e.g. from 1 to 3 heteroatoms selected from the group consisting of nitrogen, oxygen and sulfur.

Preferably, A is CH₂.

Preferably, X is NH.

Preferably, n is 0 or an integer of 1 or 2 and R⁴ is fluoro.

Preferably, R¹ is i-propyl.

Preferably, R³ is selected from the group consisting of phenyl, which may be substituted with one or two fluoro groups, and pyridyl.

Preferably, p is 0.

Preferably, A¹ and A² are absent.

Preferably, B is OR⁶ or COOR⁷.

Preferably, X is O, r is 1, A¹ is absent, A² is (CH₂)ᵥ, wherein v is 1 or 2, and B is OR⁶ or NR⁸R⁹, and R⁶, R⁸ and R⁹ are methyl.

Preferably, B is CR¹⁰═NOR¹¹R¹⁰ wherein R¹⁰ is H and R¹¹ is methyl or i-butyl or B is CONR⁸R⁹ wherein R⁸ and R⁹ are selected from the group consisting of H, methyl, ethyl and propyl, or R⁸ and R⁹, together with N, form a 5-member ring.

Preferably, A¹ is absent, r is 0, A² is CH₂ and B is OR⁶, wherein R⁶ is H, or X is O, r is 1 and B is COR¹⁰, wherein R¹⁰ is methyl.

Examples of such compounds include the following:

| NO. | Compound |
|---|---|
| 49 | 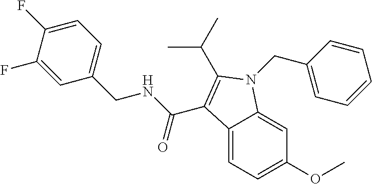 |
| 50 | 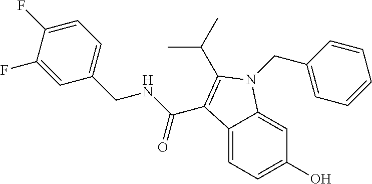 |
| 51 | 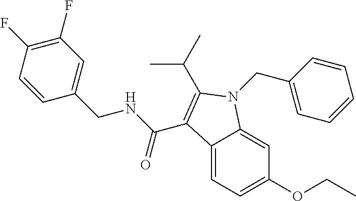 |
| 52 | 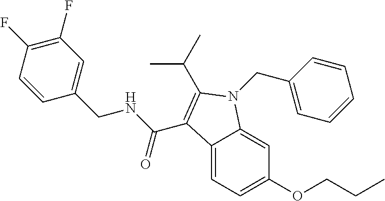 |
| 53 | 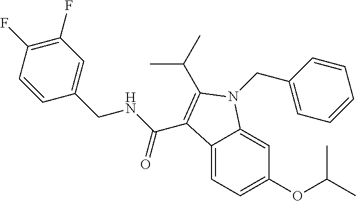 |
| 54 | 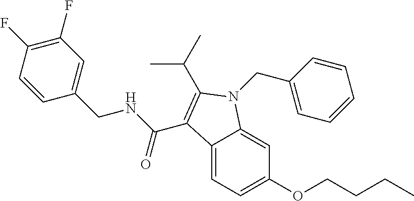 |
| 55 | 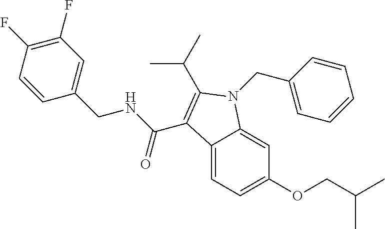 |
| 56 | 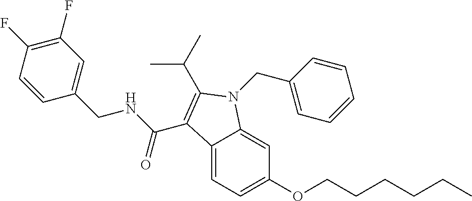 |
| 57 | 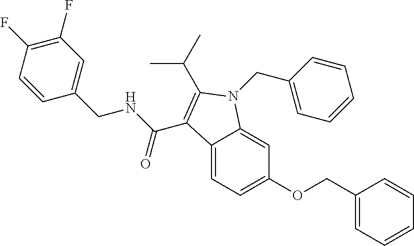 |
| 58 | 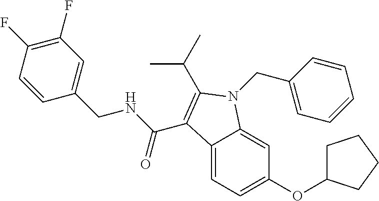 |
| 59 | 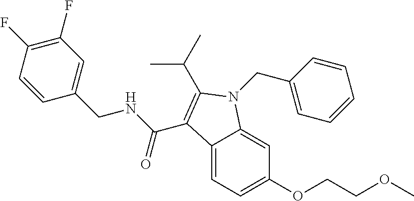 |
| 60 | 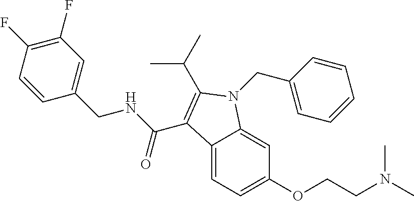 |
| 61 | 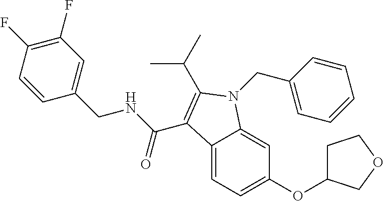 |
| 62 | 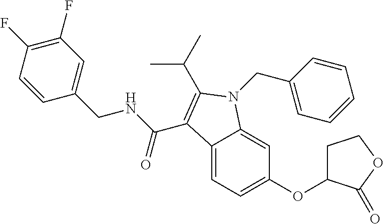 |
| 63 | 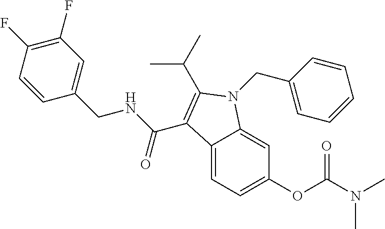 |
| 64 | 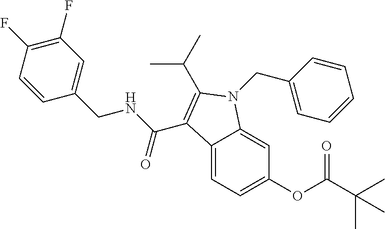 |
| 65 | 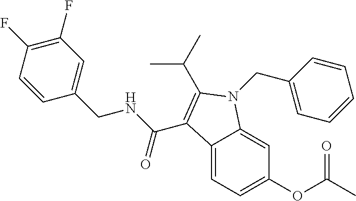 |
| 66 | 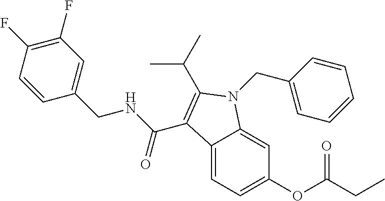 |
| 67 | 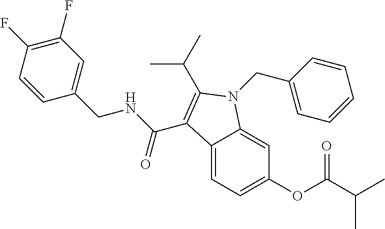 |
| 68 | 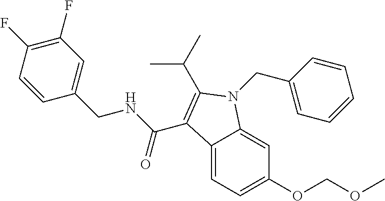 |
| 69 | 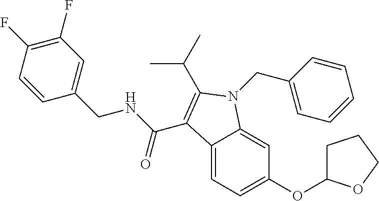 |
| 70 | 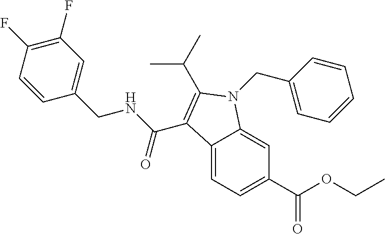 |
| 71 | 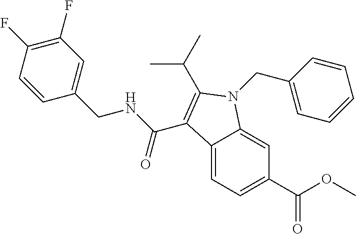 |
| 72 | 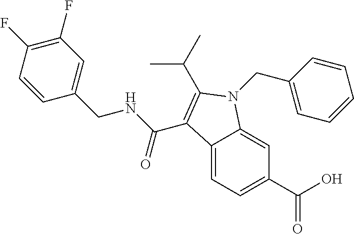 |
| 73 | 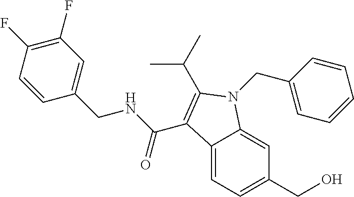 |
| 74 | 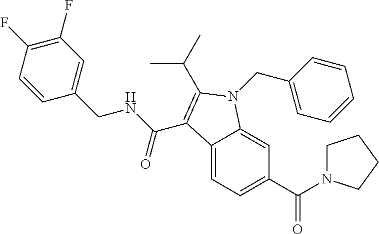 |
| 75 | 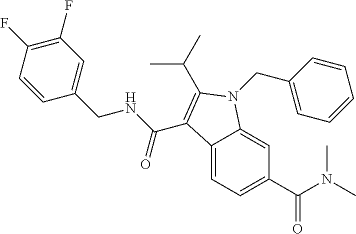 |
| 76 | 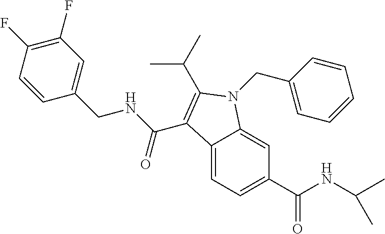 |
| 77 | 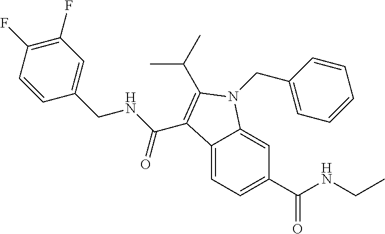 |
| 78 | 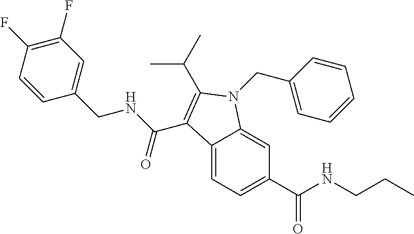 |
| 79 | 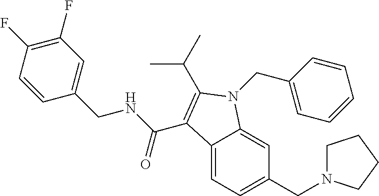 |
| 80 | 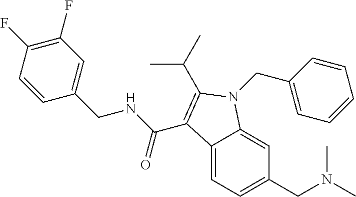 |
| 81 | 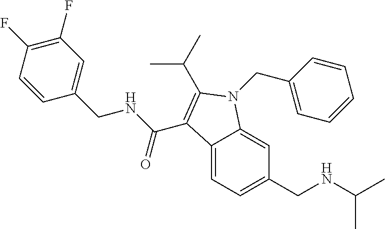 |
| 82 | 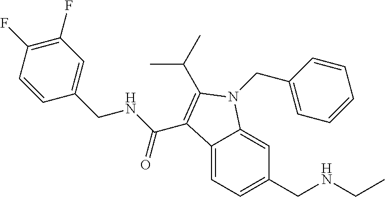 |
| 83 | 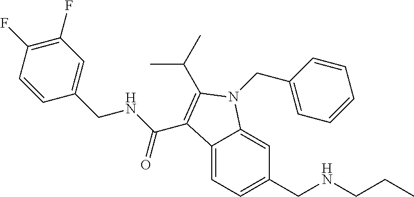 |
| 84 | 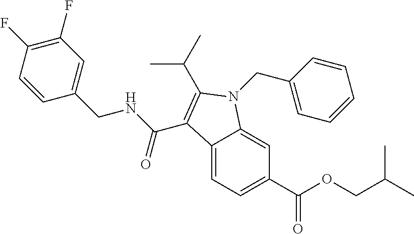 |
| 85 | 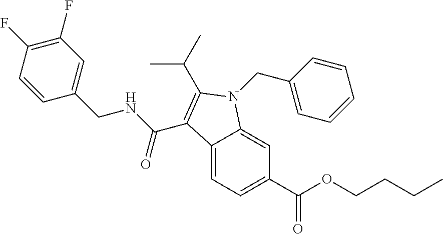 |
| 86 | 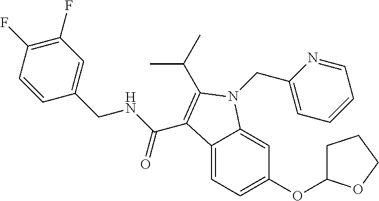 |
| 87 | 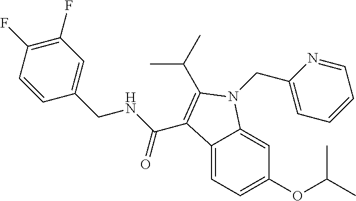 |
| 88 | 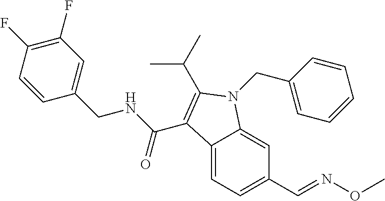 |
| 89 | 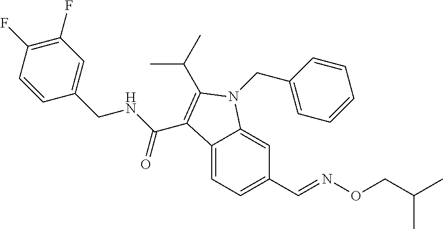 |
| 90 | 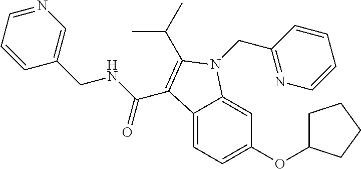 |
| 91 | 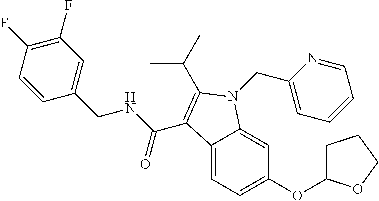 |
| 92 | 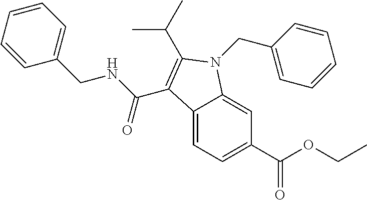 |
| 93 | 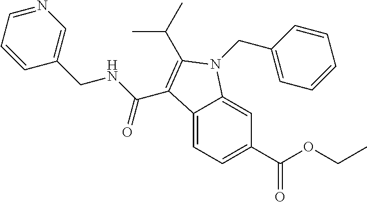 |
| 94 | 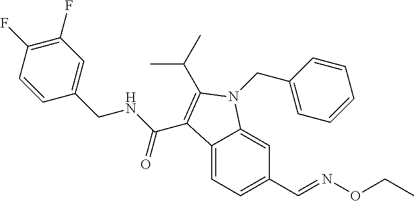 |
| 95 | 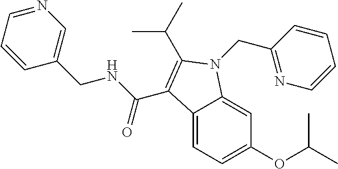 |
| 96 | 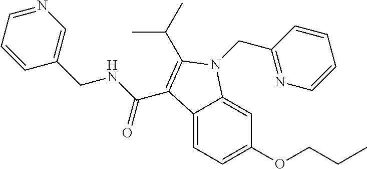 |
| 97 | 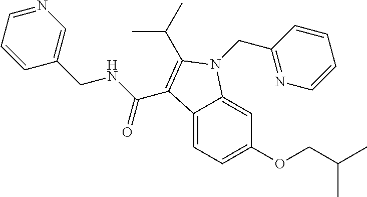 |
| 98 | 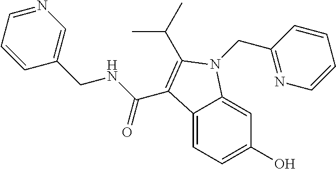 |
| 99 | 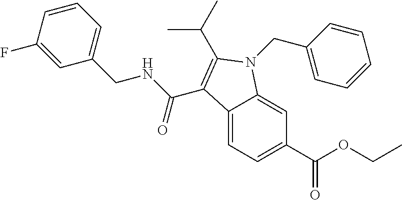 |
| 100 | 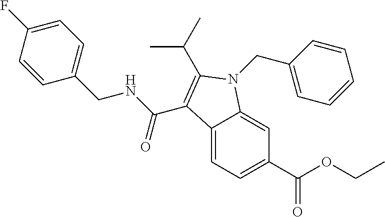 |
| 101 | 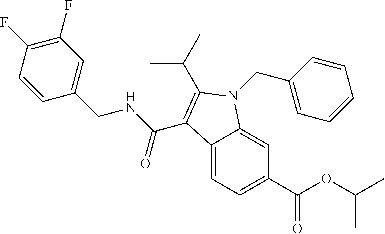 |
| 102 | 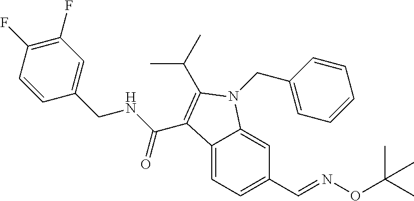 |
| 103 | 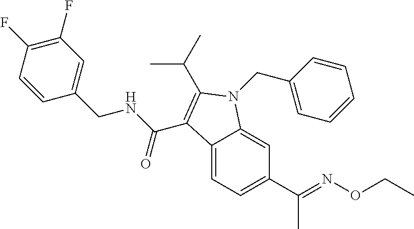 |
| 104 | 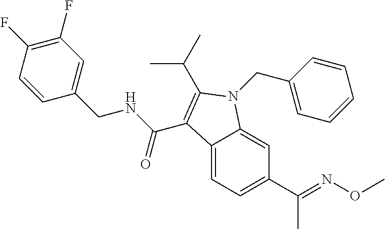 |
| 105 | 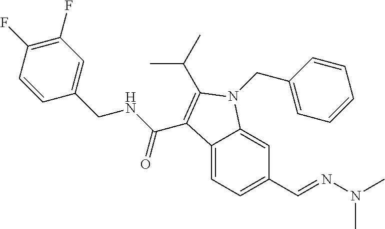 |
| 106 | 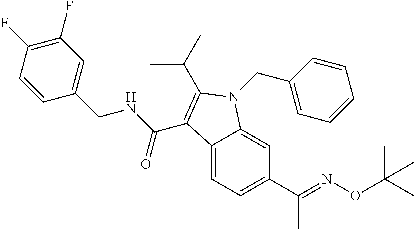 |
| 107 | 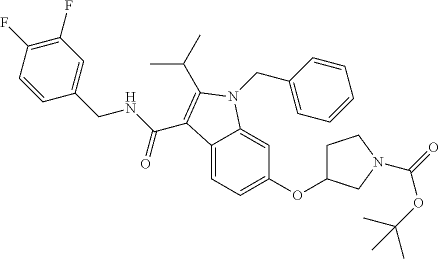 |
| 108 | 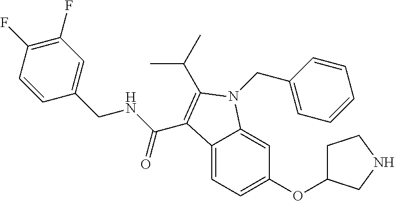 |
| 109 | 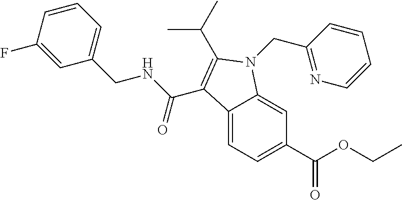 |
| 110 | 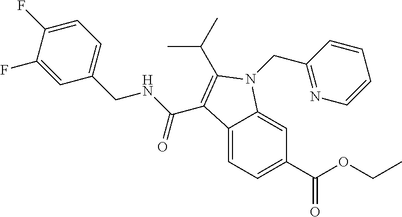 |
| 111 | 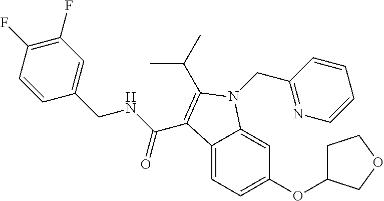 |
| 112 | 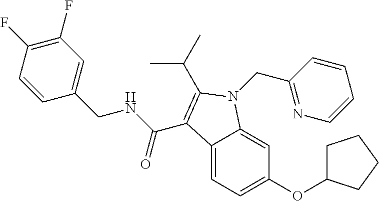 |
| 113 | 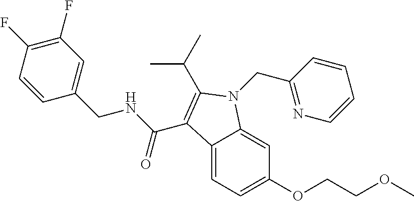 |
| 114 | 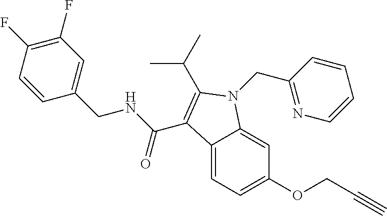 |
| 115 | 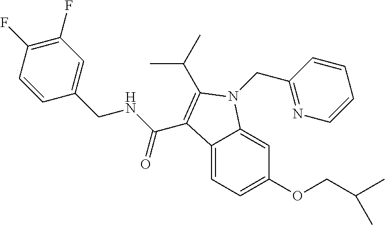 |
| 116 | 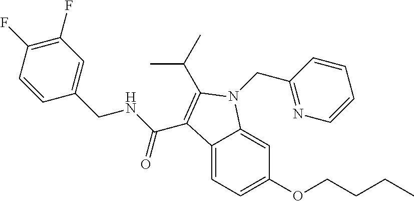 |
| 117 | 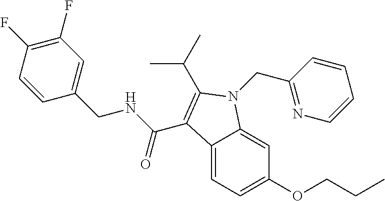 |
| 118 | 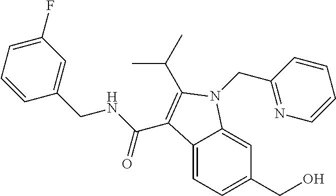 |
| 119 | 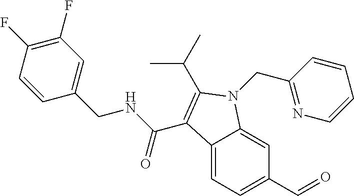 |
| 120 | 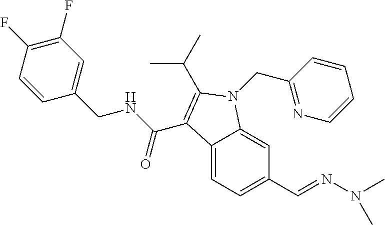 |
| 121 | 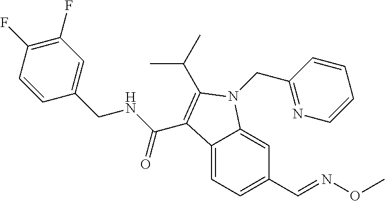 |

### Other Indole Compounds

Other compositions useful in the methods of the invention include those disclosed in U.S. patent application Ser. No. 11/690,637. That application discloses S1P3 receptor antagonists having the following formula:

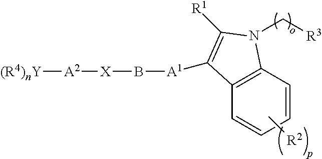

wherein:

A¹ and A² are independently selected from the group consisting of (CH₂)m where m is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

B is selected from the group consisting of (CH₂)n, where n is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, C═C(R⁵)₂, C═O, C═S, R⁵C═NR⁵, R⁵C═CR⁵, C═NOR⁵, CR⁵OR⁵, C(OR⁵)₂, CR⁵N(R⁵)₂, C(N(R⁵)₂)₂, CR⁵SR⁵, C(SR⁵)₂, SO, SO₂, and heterocyclic aryl comprising from 2 to 14 carbon atoms and from 1 to 3 heteroatoms selected from the group consisting of nitrogen, oxygen and sulfur;

X is selected from the group consisting of (CH₂)r, where r is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

provided that when m is 0 and B is C═O then X is not NR⁵, O or S;

Y is R⁶, or a carbocyclic aryl group comprising from 6 to 14 carbon atoms or a heterocyclic aryl group comprising from 2 to 14 carbon atoms and from 1 to 3 heteroatoms selected from the group consisting of nitrogen, oxygen and sulfur;

o is 0 or an integer of from 1 to 3;

p is 0 or an integer of from 1 to 4;

R¹, R², R³, R⁴ are independently selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxy, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, sulfonyl,

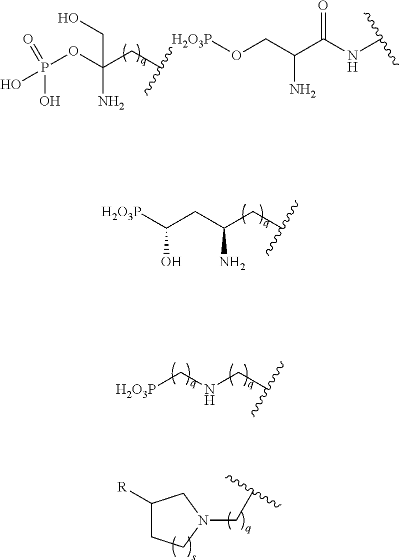

wherein R is CO₂H or PO₃H₂ and q is 0 or an integer of 1 to 5 and s is 0 or an integer from 1 to 3;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl; and

R⁶ is selected from the group consisting of straight or branched chain alkyl having 1 to 12 carbons, cycloalkyl having 3 to 6 carbons, alkenyl having 2 to 6 carbons and 1 or 2 double bonds and alkynyl having 2 to 6 carbons and 1 or 2 triple bonds.

Examples of such compounds include the following.

| NO. | COMPOUND |
|---|---|
| 122 | 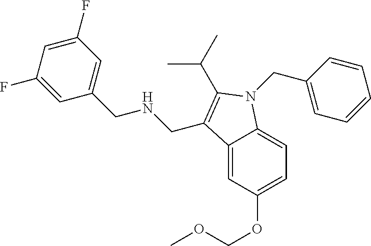 |
| 123 | 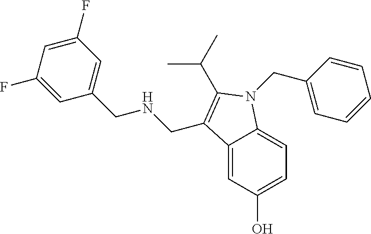 |
| 124 | 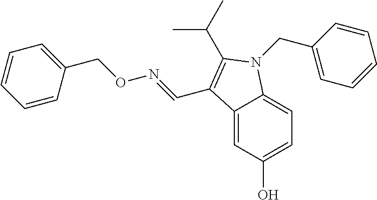 |
| 125 | 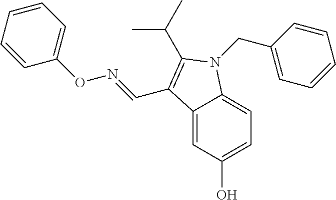 |
| 126 | 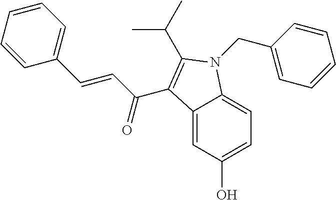 |
| 127 | 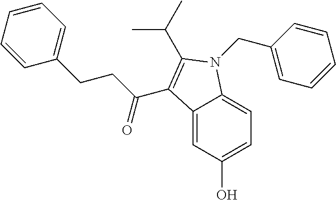 |
| 128 | 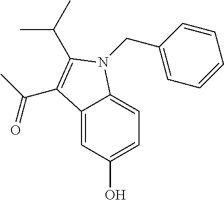 |
| 129 | 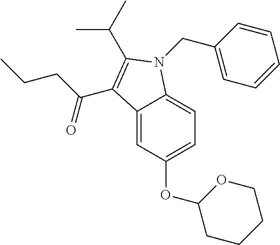 |
| 130 | 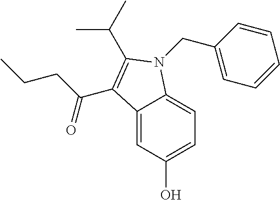 |
| 131 | 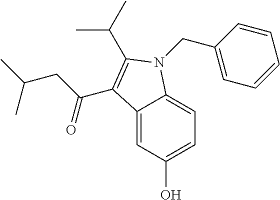 |
| 132 | 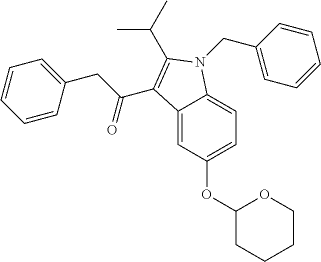 |
| 133 | 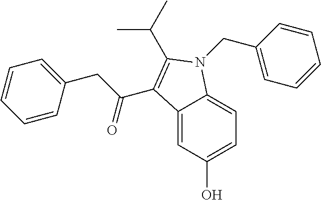 |
| 134 | 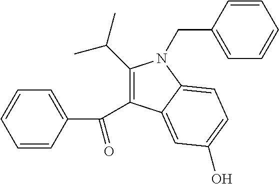 |
| 135 | 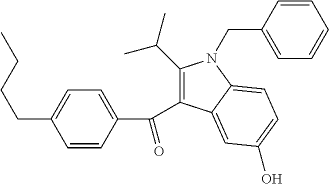 |
| 136 | 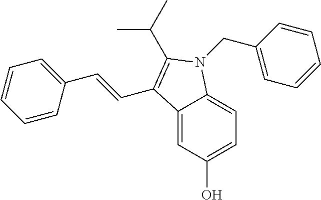 |
| 137 | 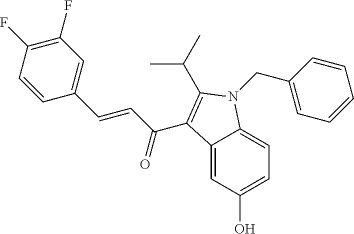 |
| 138 | 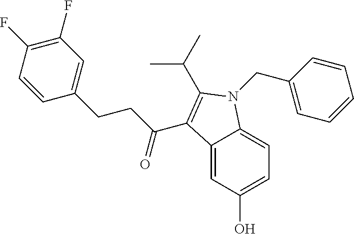 |
| 139 | 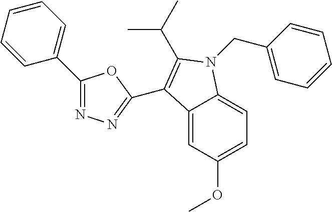 |
| 140 | 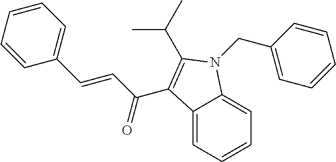 |
| 141 | 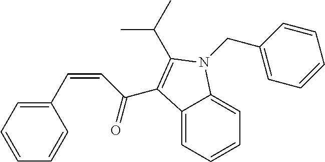 |
| 142 | 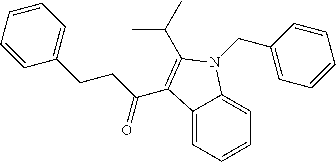 |
| 143 | 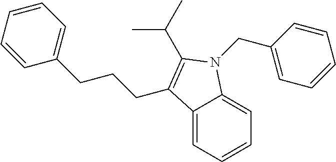 |
| 144 | 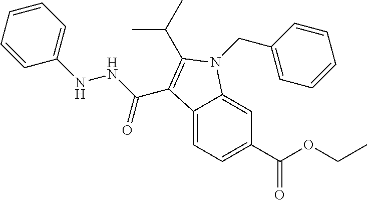 |
| 145 | 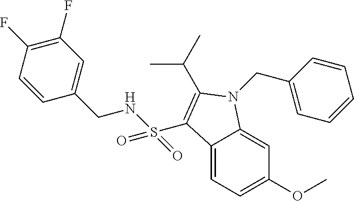 |

### Heteraromatic Compounds

Other compositions useful in the methods of the invention include those disclosed in U.S. Patent Application No. 60/824,807. That application discloses S1P3 receptor antagonists having the following formula:

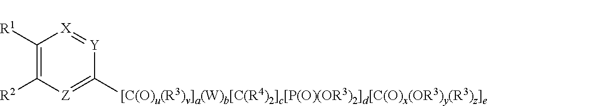

wherein

X is selected from the group consisting of CR³ and N;

Y is selected from the group consisting of CR³ and N;

Z is selected from the group consisting of CR³ and N;

at least one of X, Y and Z is N;

W is NR³ or O;

R¹ is an aryl group;

R² is an aryl group;

R³ is selected from the group consisting of H and alkyl; and 2 of said R³ groups may together with N may form a heterocylic ring having from 2 to 6 carbon atoms;

R⁴ is selected from the group consisting of H, alkyl, OR³, and N(R³)₂;

a is 0 or an integer of from 1 to 6;

b is 0 or 1;

c is 0 or an integer of from 1 to 6;

d is 0 or 1;

e is 0 or 1;

u is 0 or 1;

v is 0 or an integer of from 1 to 2;

x is 0 or 1;

y is 0 or an integer of from 1 to 3;

z is 0 or an integer of from 1 to 3;

provided, however, that when d is 0, e is 1, and when e is 0, d is 1.

Examples of such compounds include the following. Several of these selectively inhibit the S1P3 receptor subtype as compared to at least the S1P1 receptor subtypes. The EC₅₀ and IC₅₀ values expressed in the following table were obtained in the FLIPR assay described above. EC₅₀ or IC₅₀ values are stated first, followed by percent efficacy or percent inhibition stated in parenthesis. In this table and the next, percent efficacy is defined as percent of receptor activity induced by a test compound at the highest dose tested (10 μM) relative to the receptor activity induced by 5 nM sphingosine-1-phosphate, and percent inhibition is defined as percent of receptor activity induced by 5 nM sphingosine-1-phosphate that is inhibited by a test compound at the highest dose tested (10 μM). “NA” means that no activity was detected at highest dose tested; “ND” means not determined.

| | | S1P1 | S1P3 |
|---|---|---|---|
| NO. | COMPOUND | (EC50) | (IC50) |
| 146 | 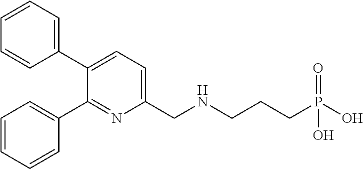 | ND | 1.6 M (83) |
| 147 | 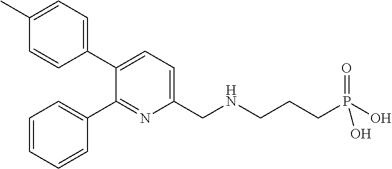 | 121 nM (36) | 231 nM (98) |
| 148 | 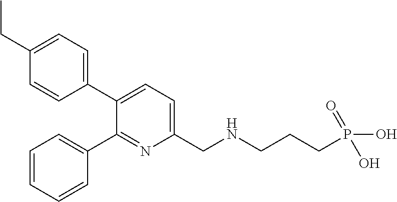 | 170 nM (57) | 319 nM (98) |
| 149 | 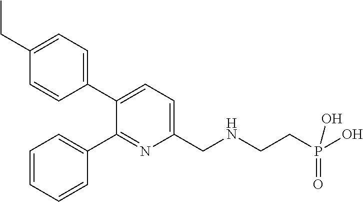 | NA | 1.8 M (99) |
| 150 | 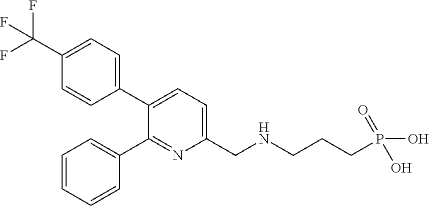 | ND | ND (95) |
| 151 | 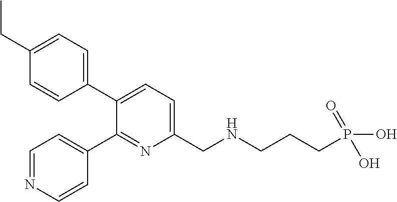 | NA | 1.1 M (95) |
| 152 | 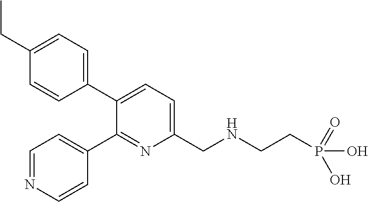 | NA | 1.8 M (68) |
| 153 | 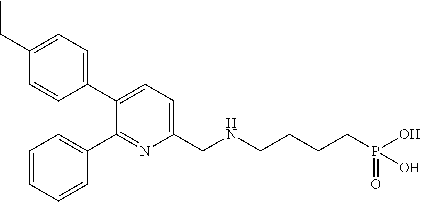 | NA | ND (30) |
| 154 | 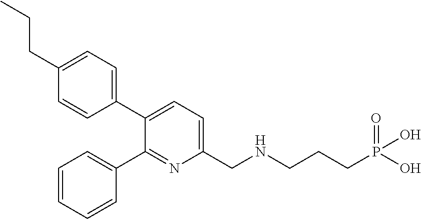 | 114 nM (69) | 319 nM (98) |
| 155 | 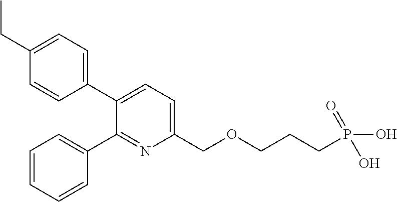 | NA | 4.0 M (27) |
| 156 | 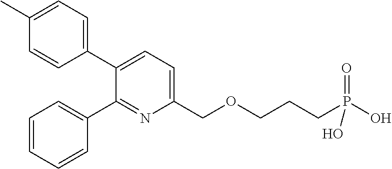 | NA | 1.9 M (11) |
| 157 | 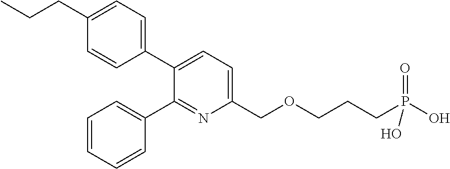 | NA | ND |

### Additional Selective S1P3 Receptor Inhibitors

Examples of compounds that selectively inhibit the S1P3 receptor subtype as compared to at least the S1P1 and S1P2 receptor subtypes include the following. The IC₅₀ values expressed below were obtained in the FLIPR assay described above. IC₅₀ values are stated first (except as otherwise noted), followed by percent efficacy or percent inhibition in parenthesis.

| | S1P1 | S1P2 | S1P3 |
|---|---|---|---|
| STRUCTURE | (IC₅₀) | (IC₅₀) | (IC₅₀) |
| 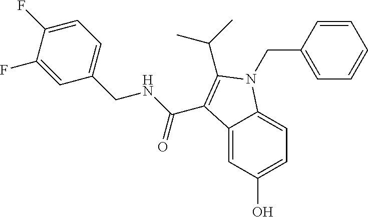 | NA | NA | 35 nM (98) |
| 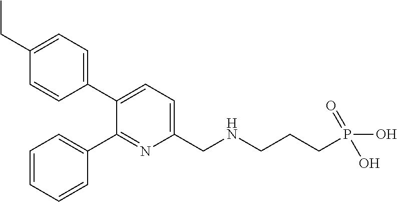 | EC₅₀ = 170 nM (57) | NA | 319 nM (98) |
| 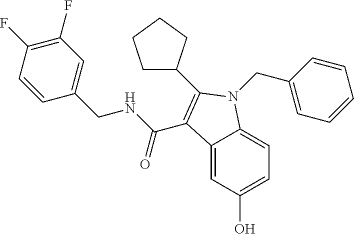 | NA | NA | 31 nM (100) |
| 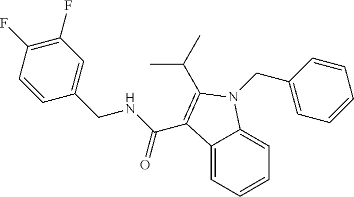 | NA | NA | 209 nM (100) |
| 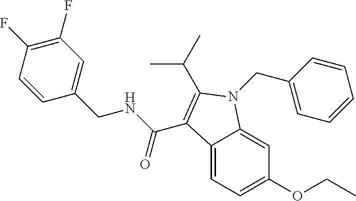 | NA | NA | 19 nM (100) |
| 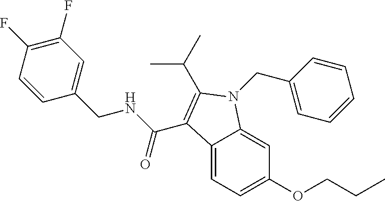 | NA | NA | 5 nM (100) |
| 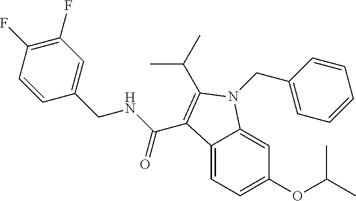 | NA | NA | 6 nM (100) |
| 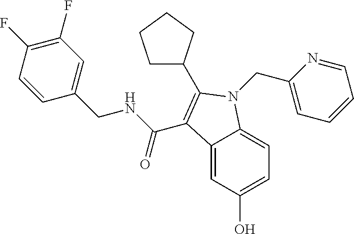 | NA | NA | 17 nM (99) |

### S1P3 Inverse Agonists

U.S. Patent Publication No. 2005/022422 discloses S1P3 receptor inhibitors that are inverse agonists of S1P3. The inhibitors have the following formula

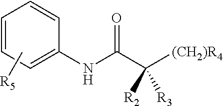

wherein R₂ is H, R₃ is NH₂, R₄ is phosphate, and R₅ is (CH₂)₇CH₃, wherein R₅ may be in the ortho or meta position.

### Thiazolidine S1P3 Antagonists

U.S. Patent Application Publication No. 2008/0025973 (the “'973 publication”) discloses S1P3 receptor inhibitors having the following structures:

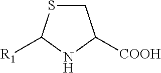

wherein R₁ is C₆-C₁₃ alkyl, or alkyl-substituted aryl where the substitution is C₅-C₉ alkyl;

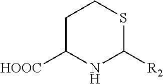

wherein where R₂ is C₉-C₁₃ alkyl; and

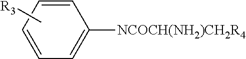

wherein R₃ is o- or m- C₅-C₈ alkyl; and R₄ is phosphate, phosphate analog, phosphonate, or sulfate. As used here, “phosphate analog” includes phosphoro-thioates, -dithioates, -selenoates, -diselenoates, -anilothioates, -anilidates, -amidates, and boron phosphates, for example.

### Pharmaceutically Acceptable Salts

One can use in the compositions and methods of the invention any S1P3 receptor inhibitor as its pharmaceutically acceptable salt.

A “pharmaceutically acceptable salt” is any salt which retains the biological effectiveness and properties of the free bases and which are obtained by reaction with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

### Prodrugs

One can use in the methods of the invention a prodrug of any of the compositions of the invention.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of C1 (i.e., the terminal carboxylic acid of a natural prostaglandin), or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

The S1P3 receptor inhibitors of the invention may be either synthetically produced, or may be produced within the body after administration of a prodrug. Hence, “S1P3 receptor inhibitor” encompasses compounds produced by a manufacturing process and those compounds formed in vivo only when another drug administered.

### Isomers and Racemates

One can use in the compositions and methods of the invention an enantiomer, stereoisomer, or other isomer of any S1P3 receptor inhibitor.

Conditions of the Eye

Conditions of the eye that may be treated with the method of the invention includes the following: conditions affecting the posterior part of the eye, such as maculopathies and retinal degeneration including non-exudative age related macular degeneration, exudative age related macular degeneration, choroidal neovascularization, diabetic retinopathy, acute macular neuroretinopathy, central serous chorioretinopathy, cystoid macular edema, and diabetic macular edema; uveitis, retinitis, and choroiditis such as acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; vasuclar diseases/exudative diseases such as retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, and bone marrow transplant retinopathy; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigement epitheliitis.

### Administration

One can use any of the compounds described above to treat conditions of the eye. To “treat,” as used here, means to deal with medically. It includes both preventing conditions of the eye and relieving symptoms associated with the conditions, whether such prevention or relief is complete or partial.

### Dose

The precise dose and frequency of administration depends on the severity and nature of the patient's condition, on the manner of administration, on the potency and pharmacodynamics of the particular compound employed, and on the judgment of the prescribing physician. Determining dose is a routine matter that is well within the capability of someone of ordinary skill in the art.

The compositions of the invention may be administered orally or parenterally, the later by subcutaneous injection, intramuscular injection, intravenous administration, or other route.

### Excipients and Dosage Forms

Those skilled in the art will readily understand that for administering pharmaceutical compositions of the invention the S1P3 receptor inhibitor may be admixed with pharmaceutically acceptable excipient which are well known in the art.

A pharmaceutical composition to be administered systemically may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, U.S. Pat. No. 4,166,452, and U.S. Pat. No. 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

1.-2. (canceled)

3. A method for treating a condition of the eye selected from the group consisting of non-exudative age related macular degeneration, exudative age related macular degeneration, choroidal neovascularization, diabetic retinopathy, acute macular neuroretinopathy, central serous chorioretinopathy, cystoid macular edema, diabetic macular edema, uveitis, retinitis, choroiditis, Behcet's disease, birdshot retinochoroidopathy, infectious uveitis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telanqiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, anqioid streaks, familial exudative vitreoretinopathy, Eales disease, sympathetic ophthalmia, uveitic retinal disease, retinal detachment, trauma, conditions caused by laser, conditions caused by photodynamic therapy, photocoaqulation, hypoperfusion during surgery, radiation retinopathy, bone marrow transplant retinopathy, proliferative vitreal retinopathy, epiretinal membranes, proliferative diabetic retinopathy; ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, myiasis, retinitis piqmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsbv's fundus dystrophy, benign concentric maculopathy, Bietti's crystalline dystrophy, pseudoxanthoma elasticum, retinal detachment, macular hole, giant retinal tear, retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors, punctate inner choroidopathy, acute posterior multifocal placoid pigment epitheliopathy, myopic retinal degeneration, and acute retinal piqement epitheliitis, the method comprising the step of administering to a patient in need of such treatment a compound represented by the general formula I: 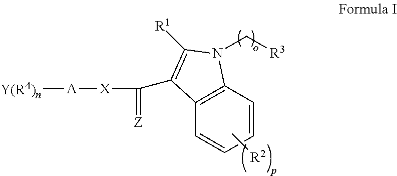
or a pharmaceutically acceptable salt thereof, wherein
X is NR⁵, O, S;
Z is O or S;
n is 0 or an integer of from 1 to 4;
o is 0 or an integer of from 1 to 3;
p is 0 or an integer of from 1 to 4;
A is (C(R⁵)₂)ₘ, wherein
m is 0 or an integer of from 1 to 6;
R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl groups;
Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;
R¹, R², R³, R⁴ are selected from the group consisting of hydrogen; straight or branched chain alkyl having 1 to 12 carbons; cycloalkyl having 3 to 6 carbons; alkenyl having 2 to 6 carbons and 1 or 2 double bonds; alkynyl having 2 to 6 carbons and 1 or 2 triple bonds; aryl wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur; halo; C₁ to C₁₂ haloalkyl; hydroxyl; C₁ to C₁₂ alkoxy; C₃ to C₂₀ arylalkyloxy; C₁ to C₁₂ alkylcarbonyl; formyl; oxycarbonyl; carboxy; C₁ to C₁₂ alkyl carboxylate; C₁ to C₁₂ alkyl amide; aminocarbonyl; amino; cyano; diazo; nitro; thio; sulfoxyl; sulfonyl groups; or a group selected from the group consisting of 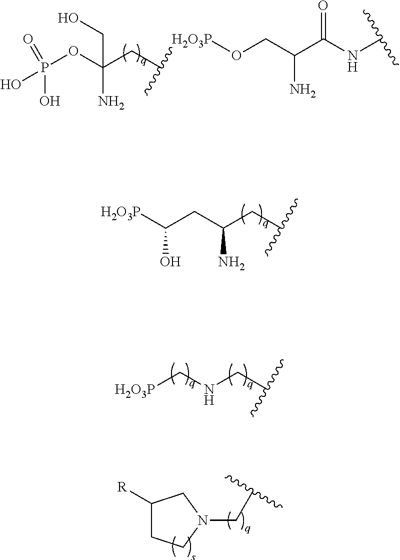
wherein R is CO₂H or PO₃H₂, p is an integer of 1 or 2 and q is 0 or an integer of 1 to 5 and s is 0 or an integer of 1 or 2; provided that, if Y is phenyl, it must be substituted with at least one R⁴ group that is not hydrogen.

4. The method of claim 3, wherein Z is O.

5. The method of claim 3, wherein Y is a phenyl group, or a heterocyclic aryl group selected from the group consisting of pyridyl, thienyl, furyl, pyradizinyl, pyrimidinyl, pyrazinyl, thiazolyl, oxazolyl, and imidazolyl.

6. The method of claim 5 wherein each said aryl is independently selected from the group consisting of phenyl, pyridine, pyrazine, pyridazine, pyrimidine, triazine, thiophene, furan, thiazole, thiadiazole, isothiazole, oxazole, oxadiazole, isooxazole, naphthalene, quinoline, tetralin, chroman, thiochroman, tetrahydroquinoline, dihydronaphthalene, tetrahydronaphthalen, chromene, thiochromene, dihydroquinoline, indan, dihydrobenzofuran, dihydrobenzothiophene, indene, benzofuran, benzothiophene, coumarin and coumarinone, wherein said aryl is unsubstituted or is substituted with one or two alkyl, alkenyl, alkynyl, aryl, halo, haloalkyl, hydroxyl, alkoxyl, alkylcarbonyl, formyl, oxycarbonyl, carboxyl, alkyl carboxylate, alkyl amide, aminocarbonyl, amino, cyano, diazo, nitro, thio, sulfoxyl, or sulfonyl groups.

7. The method of claim 4, wherein Y is phenyl.

8. The method of claim 4, wherein A is CH₂.

9. The method of claim 8, wherein X is NH.

10. The method of claim 9, wherein n is 0 or an integer of 1 or 2 and R⁴ is selected from the group consisting of methyl, methoxy, fluoro and chloro.

11. The method of claim 10, wherein R¹ is selected from the group consisting of hydrogen, methyl, ethyl and i-propyl.

12. The method of claim 8, wherein R³ is selected from the group consisting of methyl, butyl, phenyl, benzyl, pyridyl, furanylmethylenyl, thienyl and thienyl methylenyl.

13. The method of claim 12, wherein p is 0 or p is 1 and R² is selected from the group consisting of hydroxyl, methoxy, nitro, amino, acetamido and benzyloxy.

14. The method of claim 13, wherein p is 1 and R² is a 5-hydroxy group; R¹ is selected from the group consisting of methyl, ethyl, i-propyl and phenyl; R³ is selected from the group consisting of benzyl, thienylmethylenyl and furanylmethylenyl; n is 1 or 2 and R⁴ is selected from the group consisting of methoxy and fluoro.

15. The method of claim 3 wherein said compound is selected from the group consisting of
1-Benzyl-5-hydroxy-2-methyl-1H-indole-3-carboxylic Acid, 3,5-Difluorobenzylamide;
5-Hydroxy-2-methyl-1-thiophen-2-ylmethyl-1H-indole-3-carboxylic Acid, 3,4-Difluorobenzylamide;
1-Butyl-5-hydroxy-2-methyl-1H-indole-3-carboxylic Acid, 3,5-Difluoro-benzylamide;
1-Furan-2-ylmethyl-5-hydroxy-2-methyl-1H-indole-3-carboxylic Acid, 3,4-Difluorobenzylamide;
5-Hydroxy-2-methyl-1-thiophen-2-ylmethyl-1H-indole-3-carboxylic Acid, 3,5-Difluorobenzylamide;
1-Furan-2-ylmethyl-5-hydroxy-2-methyl-1H-indole-3-carboxylic Acid 3,5-Difluorobenzylamide;
1-Benzyl-5-hydroxy-2-methyl-1H-indole-3-carboxylic Acid. 3,4-Difluoro-benzylamide;
5-Hydroxy-2-methyl-1-thiophen-2-ylmethyl-1H-indole-3-carboxylic Acid, 3-Fluorobenzylamide;
5-Hydroxy-2-methyl-1-thiophen-2-ylmethyl-1H-indole-3-carboxylic Acid, Benzylamide;
5-Hydroxy-2-methyl-1-thiophen-2-ylmethyl-1H-indole-3-carboxylic Acid, 3-Methoxybenzylamide;
1-Butyl-5-hydroxy-2-methyl-1H-indole-3-carboxylic Acid, 3-Methoxy-benzylamide;
5-Hydroxy-2-methyl-1-thiophen-2-ylmethyl-1H-indole-3-carboxylic Acid, 4-Fluorobenzylamide;
5-Hydroxy-2-methyl-1-thiophen-2-ylmethyl-1H-indole-3-carboxylic Acid, 4-Methylbenzylamide;
5-Hydroxy-2-methyl-1-thiophen-2-ylmethyl-1H-indole-3-carboxylic Acid, 3-Chlorobenzylamide;
5-Hydroxy-2-methyl-1-thiophen-2-ylmethyl-1H-indole-3-carboxylic Acid, 4-Chlorobenzylamide;
5-Hydroxy-2-methyl-1-thiophen-2-ylmethyl-1H-indole-3-carboxylic Acid, 2-methoxybenzylamide;
1-Benzyl-2-ethyl-5-hydroxy-1H-indole-3-carboxylic Acid, 3,4-Difluoro-benzylamide;
1-Benzyl-2-ethyl-5-hydroxy-1H-indole-3-carboxylic Acid, 3-Methoxy-benzylamide;
1-Benzyl-5-hydroxy-2-isopropyl-1H-indole-3-carboxylic Acid, 3,4-Difluorobenzamide;
5-Hydroxy-2-methyl-1-phenyl-1H-indole-3-carboxylic Acid 3,4-Difluoro-benzylamide;
5-Hydroxy-2-methyl-1-pyridin-2-yl-1H-indole-3-carboxyl ic Acid 3,4-Difluoro-benzylamide;
5-Hydroxy-2-methyl-1-thiophen-2-yl-1H-indole-3-carboxylic Acid 3,4-Difluorobenzylamide;
1-Benzyl-2-ethyl-5-hydroxy-1H-indole-3-carboxylic Acid 3,5-Difluoro-benzylamide;
1-Benzyl-5-hydroxy-2-isopropyl-1H-indole-3-carboxylic Acid, 3,5-difluorobenzylamide;
1-Benzyl-5-hydroxy-2-isopropyl-1H-indole-3-carboxylic Acid, 3-methoxybenzylamide; and
1-Benzyl-5-hydroxy-2-phenyl-1H-indole-3-carboxylic Acid, 3,5-Difluoro-benzylamide; or a pharmaceutically acceptable salt thereof.

16.-34. (canceled)

35. A method of treating a conditions of the eye selected from the group consisting of non-exudative age related macular degeneration, exudative age related macular degeneration, choroidal neovascularization, diabetic retinopathy, acute macular neuroretinopathy, central serous chorioretinopathy, cystoid macular edema, diabetic macular edema, uveitis, retinitis, choroiditis, Behcet's disease, birdshot retinochoroidopathy, infectious uveitis, intermediate uveitis (pars planitis), multifocal choroiditis, multiple evanescent white dot syndrome (mewds), ocular sarcoidosis, posterior scleritis, serpiqinous choroiditis, subretinal fibrosis and uveitis syndrome, Vogt-Koyanagi- and Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telanqiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, anqioid streaks, familial exudative vitreoretinopathy, Eales disease, sympathetic ophthalmia, uveitic retinal disease, retinal detachment, trauma, conditions caused by laser, conditions caused by photodynamic therapy, photocoagulation, hypoperfusion during surgery, radiation retinopathy, bone marrow transplant retinopathy, proliferative vitreal retinopathy, epiretinal membranes, proliferative diabetic retinopathy; ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, myiasis, retinitis piqmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsbv's fundus dystrophy, benign concentric maculopathy, Bietti's crystalline dystrophy, pseudoxanthoma elasticum, retinal detachment, macular hole, giant retinal tear, retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors, punctate inner choroidopathy, acute posterior multifocal placoid pigment epitheliopathy, myopic retinal degeneration, and acute retinal piqement epitheliitis, the method comprising the step of administering to a patient in need of such treatment a compound represented by the general formula IV: 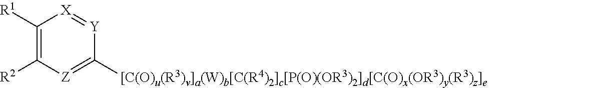
or a pharmaceutically acceptable salt thereof, wherein
X is selected from the group consisting of CR³ and N;
Y is selected from the group consisting of CR³ and N;
Z is selected from the group consisting of CR³ and N;
at least one of X, Y and Z is N;
W is NR³ or 0;
R¹ is an aryl group;
R² is an aryl group;
R³ is selected from the group consisting of H and alkyl; and 2 of said R³ groups may together with N may form a heterocylic ring having from 2 to 6 carbon atoms;
R⁴ is selected from the group consisting of H, alkyl, OR³, and N(R³)₂;
a is 0 or an integer of from 1 to 6;
b is 0 or 1;
c is 0 or an integer of from 1 to 6;
d is 0 or 1;
e is 0 or 1;
u is 0 or 1;
v is 0 or an integer of from 1 to 2;
x is 0 or 1;
y is 0 or an integer of from 1 to 3;
z is 0 or an integer of from 1 to 3;
provided, however, that when d is 0, e is 1, and when e is 0, d is 1.

36. The method of claim 35, wherein R¹ is selected from the group consisting of phenyl and substituted derivatives thereof;
R² is selected from the group consisting of phenyl, furanyl, thienyl, pyridyl, pyranyl and substituted derivatives thereof;
R³ is selected from the group consisting of H and lower alkyl;
R⁴ is selected from the group consisting of H and lower alkyl;
a is 0 or an integer of from 1 to 3; and
c is 0 or an integer of from 1 to 5.

37. The method of claim 36, wherein e is 0.

38. The method of claim 35, wherein:
R¹ is represented by the general formula 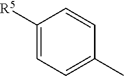
wherein R⁵ is selected from the group consisting of H, alkyl, trifluoromethyl, trifluoromethyloxy, halo and lower alkylthio;
R² is selected from the group consisting of furanyl, thienyl, pyridyl and pyranyl or R² is represented by the general formula 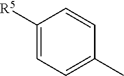
wherein R⁵ is selected from the group consisting of H, alkyl, trifluoromethyl, trifluoromethyloxy, halo, and lower alkylthio
R³ is H; and
R⁴ is selected from the group consisting of H, methyl, and ethyl.

39.-40. (canceled)

41. The method of claim 35, wherein a is 1, and c is 1, 2 or 3.

42. (canceled)

43. The method of claim 35 wherein Z is N and X and Y are CR³.

44. The method of claim 35, wherein W is NR³.

45.-50. (canceled)

51. The method of claim 35, wherein x is 1 and z is 0.

52. (canceled)

53. The method of claim 35, wherein Z is N, X and Y are CR³, R² is pyridyl, and R⁵ is selected from the group consisting of H, methyl, ethyl, propyl and trifluoromethyl.

54. The method of claim 35, wherein X, Y and Z are N, and R⁵ is selected from the group consisting of H, methyl, ethyl, propyl and trifluoromethyl.

55. The method of claim 35, wherein X and Z are N and Y is CR³.

56. The method of claim 35, wherein y is 0.

57. The method of claim 35, wherein the compound is selected from the group consisting of 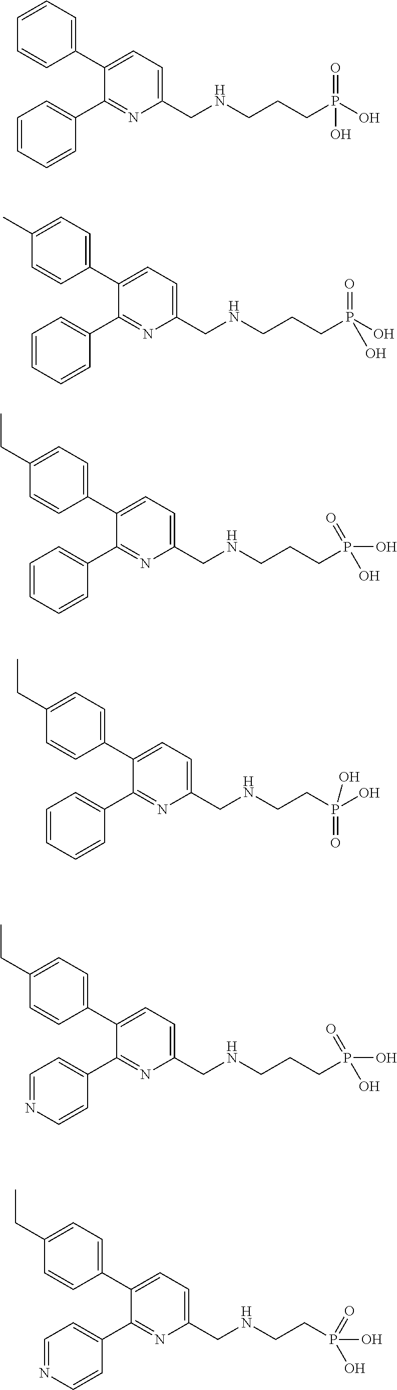 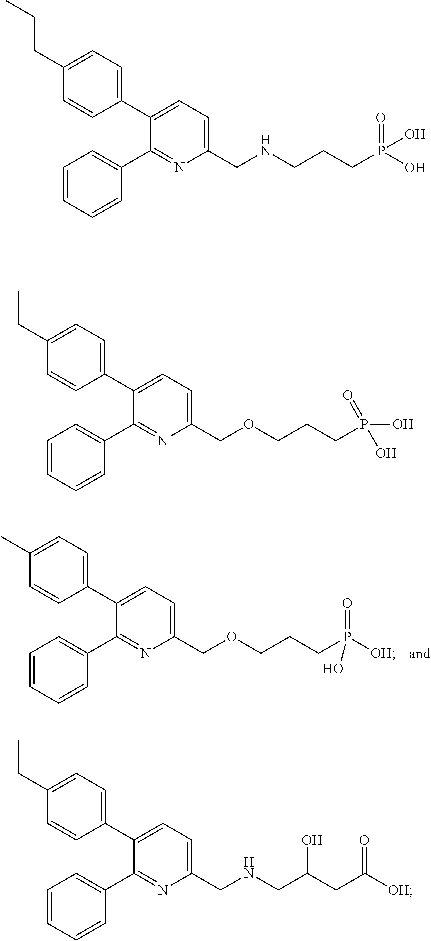
or a pharmaceutically acceptable salt thereof.

58. The method of claim 57, wherein the compounds is selected from the group consisting of 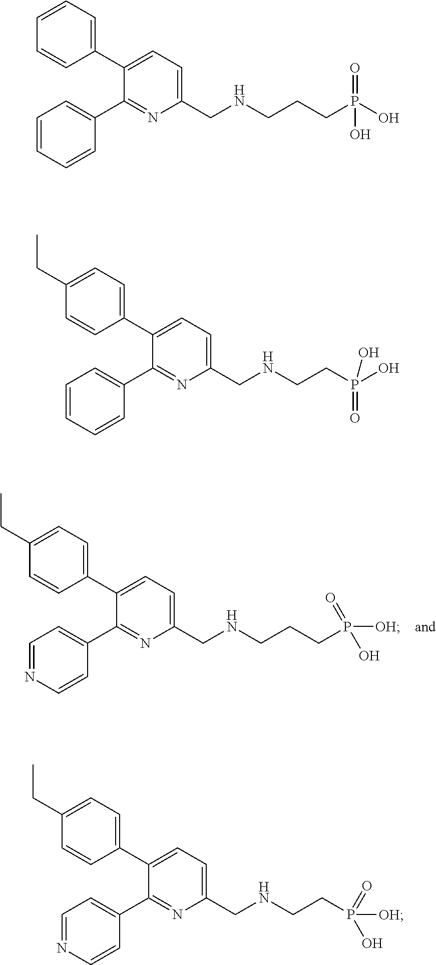
or a pharmaceutically acceptable salt thereof.

59.-67. (canceled)
